(19) United States
(12) Patent Application Publication
(10) Pub. No.: US 2010/0316622 A1
(43) Pub. Date: Dec. 16, 2010

(54) DIAGNOSIS AND TREATMENT OF CARDIAC DISORDERS

(75) Inventors: Joshua M. Hare, Miami Beach, FL (US); Daniel Gonzalez, Miami, FL (US)

Correspondence Address: NOVAK DRUCE + QUIGG LLP (WPB), 525 Okeechobee Blvd, 15th Floor, City Place Tower, West Palm Beach, FL, 33401, US

(73) Assignee: UNIVERSITY OF MIAMI, Miami, FL (US)

(21) Appl. No.: 12/740,395

(22) PCT Filed: Nov. 3, 2008
(86) PCT No.: PCT/US2008/082191

§ 371 Date: Sep. 1, 2010

Related U.S. Application Data
(60) Provisional application No. 60/984,996, filed on Nov. 2, 2007.

(51) Int. Cl. A61K 38/44; A61K 31/7088; C12Q 1/26; C12Q 1/68; C12N 5/10; C12N 15/63; A61K 31/13; A61K 31/502; A61K 38/02; A61K 31/713; A61K 31/519; G01N 33/53; A61P 9/00

(52) U.S. Cl. 424/94.4; 514/44 R; 435/25; 435/6; 435/325; 435/320.1; 514/645; 514/248; 514/16.4; 514/44 A; 514/262.1; 435/7.21

(57) ABSTRACT

Nitric oxide synthase deficiency causes diminished ryanodine receptor S-nitrosylation leading to increased diastolic calcium (Ca²+) and reduced intra sarcoplasmic reticulum calcium (Ca²+) content. Compositions for treatment of cardiac failure and cardiac disorders such as arrhythmias and sudden cardiac death are described.

### CROSS-REFERENCE TO RELATED APPLICATIONS

The present application claims the priority of U.S. provisional patent application No. 60/984,996 entitled “TREATMENT OF CARDIAC DISORDERS” filed Nov. 2, 2007, which is incorporated herein by reference in its entirety.

### GOVERNMENT SUPPORT CLAUSE

This invention was made with government support under M400-217-29998 and RO-1 HL-65455 awarded by the National Institutes of Health. The government has certain rights in the invention.

### FIELD OF THE INVENTION

Embodiments of the invention relates to the diagnosis and treatment of cardiac disorders and screening of compounds that modulate nitric oxide synthase.

### BACKGROUND

The cardiac myocyte has emerged as a prototypic example of the manner in which nitric oxide (NO) signaling occurs in a spatially confined manner. Although neuronal (NOS1) and endothelial (NOS3) isoforms of nitric oxide synthase are located extremely close to one another within the cell on opposite sides of the dyad, they exert opposite effects on myocardial contractility. The mechanism(s) for this effect remain extremely controversial. Whether this effect is mediated by a direct protein post-translational modification, is controversial.

### SUMMARY

Nitric oxide synthase (NOS) deficiency causes diminished ryanodine receptor (RyR) S-nitrosylation, which in turn lead to increased diastolic Ca²⁺ and reduced intra Sarcoplasmic Reticulum (SR) Ca²⁺ content. This leakage further lead to decreased contractility and increased electrical instability, key features of heart failure. Together these findings establish the importance of endogenous RyR2 S-nitrosylation mediated by NOS and provide mechanistic insights whereby NOS deficiency leads to both depressed myocardial contractility as well as to sudden cardiac death. Compositions for treating cardiac diseases and disorders, as well as methods for identifying these drugs, the diagnosis of subjects at risk of developing or prognosis and diagnosis of recovery of such patients will prevent both the high costs of treating such cardiac disorders, and more importantly, saving the lives of subjects.

In a preferred embodiment, a method of treating heart disease and heart disorders comprising: administering to a patient an agent comprising a nitric oxide (NO) donor or functional nitric oxide synthase molecule in a pharmaceutical composition, wherein the agent modulates ryanodine receptor (RyR) S-nitrosylation to normal levels as compared to a normal subject.

In another preferred embodiment, the administration of the NO donor comprising agent stabilizes the diastolic calcium (Ca²⁺) levels to normal levels as compared to a normal subject. Preferably, the agent is a nucleic acid molecule expressing a functional nitric oxide synthase in a patient or cardiac cells thereof. Other preferred embodiments include, without limitation, proteins, peptides, organic molecules, in organic molecules, small molecules, NO donors, NO enhancers or NO adducts or combinations thereof.

In another preferred embodiment, a method of diagnosing a subject for nitric oxide synthase (NOS) deficiency comprises obtaining a sample from a patient; and, screening the patient sample for nitric oxide synthase deficiency.

In another preferred embodiment, the patient sample is screened for levels of nitric oxide synthase, genetic differences in nitric oxide synthase nucleic acids, expression of nitric oxide synthase proteins and peptides and differences in peptide sequences as compared to a normal patient.

In another preferred embodiment, the nitric oxide deficiency screening further comprises measuring ryanodine receptor (RyR) S-nitrosylation levels in a patient as compared to a normal subject.

In another preferred embodiment, ryanodine receptor nitrosylation is measured by a biotin switch assay. However, many other assays can be used as described below.

In another preferred embodiment, a method of screening and identifying agents which modulate nitric oxide in a cell or a sample from a patient comprises culturing a nitric oxide synthase deficient cell with a candidate agent; measuring levels of nitric oxide and/or S-nitrosylation of ryanodine receptor; and, comparing the levels of nitric oxide and/or S-nitrosylation of the ryanodine receptor to a normal cell.

In another preferred embodiment, the nitric oxide synthase is neuronal nitric oxide synthase (NOS1).

In another preferred embodiment, the agents modulate NADPH oxidase (NOX) activity and xanthine oxidoreductase (XOR) activity. In one aspect of the invention, the agents inhibit NADPH oxidase (NOX) activity and/or xanthine oxidoreductase (XOR) activity.

In another preferred embodiment, a method of screening and identifying agents which modulate nitric oxide in an animal comprises administering to the animal a candidate agent; measuring levels of nitric oxide and/or S-nitrosylation of ryanodine receptor in a cell or sample from an animal; and, comparing the levels of nitric oxide and/or S-nitrosylation of the ryanodine receptor to a normal cell or sample.

In another preferred embodiment, the animal is a neuronal nitric oxide synthase (NOS1) deficient animal.

In another preferred embodiment, the candidate agent stabilizes the diastolic to calcium (Ca²⁺) levels to normal levels as compared to a normal subject or animal.

In another preferred embodiment, the candidate agents inhibit NADPH oxidase (NOX) activity and xanthine oxidoreductase (XOR) activity. Preferably, the candidate agents equilibrate the nitroso-redox levels in a cell or patient to levels compared to a normal cell.

In another preferred embodiment, a cell expresses a defective nitric oxidase synthase gene product or is deficient in nitric oxide synthase. The cell is preferably a mammalian cell and comprises a vector expressing a defective nitric oxide synthase gene product wherein nitric oxide synthase activity is decreased as compared to a normal cell. In another preferred embodiment, the cell does not produce any nitric oxide synthase.

In another preferred embodiment, a vector comprises an inducible promoter operably linked to a polynucleotide comprising at least one of nitric oxide synthase polynucleotide, ryanodine receptor, NADPH oxidase, xanthine oxidoreductase, variants, mutants and fragments thereof.

In another preferred embodiment, an isolated cell lacks a nitric oxide synthase gene product.

In another preferred embodiment, the S-nitrosylation levels of the ryanodine receptor is hyper S-nitrosylated or hypo S-nitrosylated as compared to a normal cell.

In another preferred embodiment, a method of treating cardiac arrhythmia or sudden cardiac death comprises administering to a patient an agent comprising an NO donor in a pharmaceutical composition, wherein the agent modulates ryanodine receptor (RyR) S-nitrosylation to normal levels as compared to a normal subject.

In another preferred embodiment, the administration of the NO donor comprising agent stabilizes the diastolic to calcium (Ca²⁺) levels to normal levels as compared to a normal subject and prevents the leakage of calcium.

In another preferred embodiment, a stem cell comprises a nucleic acid molecule expressing a functional nitric oxide synthase gene product. Preferably, the nitric oxide synthase gene product modulates ryanodine receptor (RyR) S-nitrosylation to normal levels as compared to a normal heart cell. In a preferred embodiment, the nitric oxide synthase gene product stabilizes the diastolic to calcium (Ca²⁺) levels to normal levels as compared to a normal heart cell and prevents the leakage of calcium.

In another preferred embodiment, the nitric oxide synthase gene product inhibits NADPH oxidase (NOX) activity and xanthine oxidoreductase (XOR) activity.

In another preferred embodiment, a method of preventing or treating a cardiac disease or disorder in a patient comprises administering to a patient a composition comprising at least one of a modulator of NADPH oxidase activity, xanthine oxidoreductase activity or nitric oxide donor in a therapeutically effective concentration.

In another preferred embodiment, the nitric oxide donor comprises nitric oxide enhancing agents, cyclic nitric oxide donors, heterocyclic nitric oxide donors, homocyclic nitric oxide donors, nitric oxide adducts, or substitutes, derivatives and variants thereof.

In another preferred embodiment, the nitric oxide donor is diethylenetriamine/nitric oxide (DETA/NO).

In another preferred embodiment, the NADPH oxidase activity modulator comprises hydralazine, nitroglycerin peptides, organic compounds, inorganic compounds, polynucleotides, oligonucleotides, proteins, antisense molecules, siRNA, or small molecules.

In another preferred embodiment, the xanthine oxidoreductase activity modulator comprises nitroglycerin, allopurinol, peptides, organic compounds, inorganic compounds, polynucleotides, oligonucleotides, proteins, antisense molecules, siRNA, or small molecules.

In another preferred embodiment, the composition comprises hydralazine, nitroglycerin and diethylenetriamine/nitric oxide (DETA/NO) therapeutically effective concentration. Preferably, the ryanodine receptor is endogenously nitrosylated.

In another preferred embodiment, the composition decreases reactive oxygen intermediates (ROS) and peroxynitrite (ONOO⁻) molecules as compared to a normal control.

In another preferred embodiment, a composition for treating cardiac disorders comprises hydralazine, nitroglycerin and diethylenetriamine/nitric oxide (DETA/NO) in a therapeutically effective concentration.

In another preferred embodiment, a method of diagnosing a patient having nitric oxide deficiency comprising obtaining a sample from a patient; screening the sample for detection of nitric oxide or nitric oxide synthase; and, determining the levels of nitric oxide in the sample as compared to a normal control.

In another preferred embodiment, the levels of nitric oxide and nitric oxide synthase are determined by methods comprising one or more of: immunoassays, Western blotting, PCR, hybridization assays, nitric oxide detection assays, electrochemical assays, fluorometric and colorimetric assays, or electroluminescent assays. Preferably, the step of detecting and determining levels of nitric oxide or nitric oxide synthase comprises measuring S-nitrosylation of a ryanodine receptor as compared to a control.

In another preferred embodiment, the ryanodine receptor is ryanodine receptor 2.

In another preferred embodiment, a method of diagnosing a patient suffering from a disorder associated with nitric oxide deficiency comprises obtaining a sample from a patient; and, assessing levels of nitrosylation of a ryanodine receptor as compared to a normal control. Preferably, nitrosylation of the ryanodine receptor is decreased as compared to a normal control.

In another preferred embodiment, a decrease in S-nitrosylation of a ryanodine receptor 2 is diagnostic of a patient suffering from a disorder associated with nitric oxide deficiency.

In another preferred embodiment, a disorder associated with nitric oxide deficiency comprises cardiac diseases, cardiac disorders or cardiac arrest.

Other aspects are discussed infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A to 1E show the Force-Frequency relationship and parameters of Ca²⁺ removal. FIG. 1A shows representative traces of sarcomere shortening and calcium transients for wild type and NOS1-deficient myocytes. FIG. 1B is a graph showing sarcomere shortening and the amplitude of [Ca²⁺]ᵢ transients (F—F₀) in response to increased frequency of stimulation is impaired in NOS1^{-/-} and NOS3^{-/-} NOS3^{-/-} but not in NOS3^{-/-} myocytes. FIG. 1C is a graph showing calibrated values of systolic and diastolic [Ca²⁺]ᵢ. NOS1^{-/-} and NOS1/NOS3^{-/-} myocytes showed increased diastolic levels of [Ca²⁺]ᵢ. ** p<0.05. FIG. 1D is a graph showing the frequency-dependent acceleration of [Ca²⁺]ᵢ decay which was estimated as a constant of decay (τ) and TR50 (time to reach 50 of baseline values). Both parameters were not significantly different among strains. FIG. 1E shows NCX function. Similar parameters as in FIG. 1A were used to analyze the function of the sodium calcium exchanger NCX, during a pulse of caffeine infusion. No significant differences between WT and NOS1 were found.

FIGS. 2A-2D show the assessment of SR calcium leak and Load-leak relationship. FIG. 2A: Original tracings showing the protocol for Ca²⁺ leak. After stimulating the myocytes (4 Hz in the example), the solution is changed to a Na—Ca free medium during approximately 40 s. After recovering, the cells are challenged again at the same frequency, and the Na—Ca free medium contains tetracaine, a RyR blocker. FIG. 2B: Intra SR Ca²⁺ content as a function of frequency of stimulation. The increased frequencies induce increase in the Ca²⁺ load, effect. FIG. 2C: Shift in intra SR content (change in cytosolic Ca with tetracaine) as a function of the load. Despite a decreased load of Ca²⁺ into the SR, the tetracaine-induced shift is increased in NOS1^{-/-} myocytes compared to the wild type, indicating increased diastolic leak. FIG. 2D: Fractional release of Ca²⁺ after stimulation of the cells at 4 Hz and a pulse of caffeine (10 mM) (n=25 wt, 30 NOS1^{-/-}). * p≦0.005.

FIGS. 3A-3C show arrythmogenic Ca²⁺ waves in NOS1 deficient myocytes. FIG. 3A: Original traces of wild type, NOS1^{-/-}, NOS1/NOS3^{-/-} and NOS3^{-/-} myocytes stimulated at 4 Hz. After a pause in 0Na⁺0Ca²⁺ buffer, intracellular Ca²⁺ waves appeared in NOS1−/− and NOS3/NOS1^{-/-} cells. These were abolished with the presence of tetracaine. FIG. 3B: Quantification of spontaneous Ca²⁺ waves/cell displayed by wt (n=16), NOS1−/− (n=12), NOS3^{-/-} (n=14) and NOS1/−/− cardiomyocytes (n=7) stimulated at 4 Hz * p<0.05. FIG. 3C: Analysis of spontaneous Ca²⁺ waves displayed by wt and NOS1^{-/-} cardiomyocytes stimulated from 1 to 6 Hz * p<0.05, ** p<0.01.

FIGS. 4A-4B show pharmacological manipulations with NOS1 blocker and NO donor. FIG. 4A: Wild type myocytes (C57BL6) were treated with the specific NOS1 inhibitor S-MTC (1 μM). After 15 mm, the cells were challenged with the Na⁺—Ca²⁺ free buffer and after 40 s, with a pulse of caffeine. FIG. 4B: NOS1^{-/-} cells were treated with an NO donor (DETA/NO, 100 μM) and then challenged with the Na—Ca free and caffeine to assess intra SR Ca²⁺ content. (n=6)* p<0.05.

FIGS. 5A-5C show stoichiometry of FKBP12.6 to RyR. FIG. 5A shows Western blots of RyR and FKBP12.6. Heart homogenates of control and NOS1^{-/-} (n=7) mice were analyzed for RyR2, and FKBP12.6. * p<0.05. FIG. 5B: The binding of FKBP to RyR was further analyzed by-co-immunoprecipitation in both strains using anti a RyR2 antibody (n=4). FIG. 5C: Phosphorylation status of RyR was also measured using a specific antibody against the phosphorylated Ser 2908 and compared to the total amount of RyR2 (n=4).

FIGS. 6A-6B show the analysis of RyR2 S-nitrosylation. FIG. 6A: Ryanodine receptor nitrosylation assed by the biotin switch. Hearts of wild type and NOS1^{-/-} mice were submitted to the biotin switch in the presence or absence of sodium ascorbate, as control. The top panel indicates the signal using anti-biotin antibody. The middle panel shows the total RyR2. The lower panel shows GAPDH as marker of protein load. The ratio of the biotin signal to the total RyR2 was compared (n=5, *p<005). FIG. 6B: Labeled proteins from the biotin switch were further purified with streptavidin-agarose and immunoblotted for RyR2 and GAPDH.

FIGS. 7A-7C show sarcomere length (SL) and Ca²⁺ transients ([Ca²⁺]ᵢ) in NOS 1^{-/-} ventricular myocytes. FIG. 7A: show representative traces of sarcomere shortening and [Ca²⁺]ᵢ in wild type and NOS 1 deficient myocytes (with or without nitroglycerin and hydralazine) stimulated at 4 Hz. FIGS. 7B, 7C: Frequency-induced increase in myocyte contractility and [Ca²⁺]ᵢ were attenuated in NOS1^{-/-} compared to WT. **P<0.01 vs WT.

FIGS. 8A-8D: FFR of SL and [Ca²⁺]ᵢ in isolated cardiac myocytes from NOS1^{-/-} mice with and without hydralazine, nitroglycerin and DETA/NO. Sarcomere shortening (SL) and [Ca²⁺]ᵢ in the presence of hydralazine (0.001 mmol/L), nitroglycerin (0.01 mmol/L) and DETA/NO (0.1 mmol/L) in wt (FIG. 8A) and in NOS1^{-/-} cells (FIG. 8B). Combination of hydralazine plus nitroglycerin and hydralazine plus DETA/NO augmented contractility and Ca²⁺ transients of NOS1^{-/-} myocytes as much as each drug alone (FIGS. 8C, 8D) (* P<0.05 and ‡ P<0.01 vs control).

FIGS. 9A-9B: Sarcoplasmic reticulum Ca²⁺ stores at 1 and 4 Hz. Isolated myocytes were rapidly exposed to caffeine (10 mmol/L) at 1 and 4 Hz after 10-second pause. FIG. 9A: Sample transients depict the SR Ca²⁺ stores at 1 and 4 Hz. Calcium stores are expressed as percent change from baseline (FIG. 9B). NOS1^{-/-} had less sarcoplasmic Ca²⁺ stores than WT at 4 Hz (‡P<0.001). Hydralazine (Hyd), nitroglycerin (nitro) and DETA/NO (D/NO) increased Ca²⁺ reserves of NOS1−/− myocytes at 4 Hz (*P<0.05 for each drug).

FIG. 10: Assessment of diastolic Ca²⁺ leak. SR Ca²⁺ leak was estimated using tetracaine (1 mmol/L) after a pause (40 s) in the pacing of the cells. After this pause (40 s), intra SR Ca²⁺ was released using caffeine. NOS1^{-/-} myocytes displayed increased Ca²⁺ leak, and also, spontaneous Ca²⁺ waves. Both parameters were significantly decreased by the treatment combination of nitroglycerin (100 μmol/L) and hydralazine (1 mmol/L) (* P<0.05).

FIG. 11: NOX-dependent O₂.⁻ production in cardiac homogenates. Effects of hydralazine and nitroglycerin on superoxide levels in the enzymatic system containing 0.3 mmol/L as substrate (for NADPH oxidase), and NOS1^{-/-} cardiac homogenates as source of the enzyme (* P<0.05 and ‡ P<0.01 vs. control).

FIGS. 12A-12D: Xanthine oxidase activity in cardiac homogenates. FIG. 12A: NOS1^{-/-} cardiac homogenates exhibited increased XOR activity versus WT mice in terms of O₂.⁻ production (*P<0.05 vs Wt). FIG. 12B shows the effect of allopurinol, hydralazine and nitroglycerin on XOR-superoxide production in NOS1−/− homogenates. FIG. 12C: NOS1^{-/-} cardiac homogenates show increased XOR-dependent uric acid production. FIG. 12D shows the effects of allopurinol, hydralazine and nitroglycerin on uric acid production in the enzymatic system containing 0.2 mmol/L xanthine as substrate (for XOR), and NOS 1^{-/-} cardiac homogenates as source of the enzyme (* P<0.05, ‡ P<0.01 vs control values).

FIGS. 13A-13D show NOX and XOR dependent peroxynitrite production in NOS1^{-/-} mice cardiac homogenates. FIG. 13A shows WT and NOS1^{-/-} heart homogenates showed similar level of NOX-dependent ONOO⁻ production. FIG. 13B shows the effect of nitroglycerin and hydralazine on the ONOO⁻ production in the enzymatic ONOO⁻ generating system containing 0.3 mmol/L NADPH as substrate (for NADPH oxidase) in NOS1^{-/-} cardiac homogenates, as source of the enzyme. FIG. 13C shows WT and NOS1^{-/-} heart homogenates showed similar level of XOR dependent ONOO⁻ production. FIG. 13D shows the effect of nitroglycerin, hydralazine and allopurinol on the ONOO⁻ production in the enzymatic system containing 0.1 mmol/L xanthine as substrate (for xanthine oxidase) and NOS1^{-/-} cardiac homogenates as a source of the enzyme (* P<0.05 and ‡ P<0.01 vs. control).

### DETAILED DESCRIPTION

We have shown that nitric oxide is important in maintaining electrical stability of the heart. Mice lacking a specific NO synthase exhibit a major disruption in the fundamental process of cardiac excitation-contraction coupling. This disruption manifests as a leak of calcium within cardiac myocytes from the sarcoplasmic reticulum (SR) into the cytoplasm of the cell. This leak is responsible for diminished cardiac contractile function.

The current approach to the treatment of patients with heart failure linked to oxidative stress mechanisms lacks individualization and becomes increasingly important in the future, as the number and classes of medicine for heart failure increase. The ability to distinguish lack of neural nitric oxide synthase in a heart patient will be a crucial component for making decisions about therapy, as well as aid in the detection of high risk patients and their tight monitoring.

In embodiments of the invention, diagnosis, prognosis of recovery and treatment of cardiac disorders and diseases provide individualized treatment protocols and the monitoring of patients.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, “cardiac disorders” or “heart disease” include cardiac or cardiovascular diseases. Examples of cardiac disorders include, but are not limited to, cardiac arrhythmia, myocardial ischemia, myocardial infarction, congestive heart failure, dilated and hypertrophic cardiomyopathy, cardiac hypertrophy, cardiac transplantation and rejection, allograft rejection (cardiac), coronary angioplasty, cardiopulmonary by-pass surgery and electrophysiological studies, restenosis, atherosclerosis, atherogenesis, angina, (particularly chronic, stable angina pectoris), ischemic disease, congestive heart failure or pulmonary edema associated with acute myocardial infarction, thrombosis, controlling blood pressure in hypertension (especially hypertension associated with cardiovascular surgical procedures), thromboembolic events, platelet aggregation, platelet adhesion, smooth muscle cell proliferation, vascular complications associated with the use of medical devices, wounds associated with the use of medical devices, cerebrovascular ischemic events, and the like. Complications associated with the use of medical devices may occur as a result of increased platelet deposition, activation, thrombus formation or consumption of platelets and coagulation proteins. Such complications, which are within the definition of “cardiovascular disease or disorder,” include, for example, myocardial infarction, ischemic stroke, transient ischemic stroke, thromboembolic events, pulmonary thromboembolism, cerebral thromboembolism, thrombophlebitis, thrombocytopenia, bleeding disorders and/or any other complications which occur either directly or indirectly as a result of the foregoing disorders.

The term “cardiac arrhythmia” is used herein to denote conditions in a mammal where there is an irregular heart action caused by physiological or pathological disturbances in discharge of cardiac impulses or their transmission through the conducting tissue of the heart, and includes tachycardia and fibrillations of particular cardiac sites, including but not limited to, atrial, essential, nodal, paroxysmal atrial, paroxysmal nodal, paroxysmal ventricular, polymorphic ventricular, reflex and sinus and ventricular fibrillation.

The term “cardiovascular agent” or “candidate therapeutic agents” refers to agents which have the potential to treat, ameliorate, or prevent disorders relating to the heart and the blood vessels or circulation. Cardiovascular agents include, for example, vasoprotective agents, antihypertensive agents, cardiomyopathy therapeutic agents, coronary heart disease therapeutic agents, and heart failure therapeutic agents.

“Medical device” refers to any intravascular or extravascular medical devices, medical instruments, medical product, foreign bodies including implants and the like, having a surface that comes in contact with tissue, blood or bodily fluids in the course of its use or operation. Examples of intravascular medical devices and instruments include balloons or catheter tips adapted for insertion, prosthetic heart valves, sutures, surgical staples, synthetic vessel grafts, stents (e.g. Palmaz-Schatz, Wiktor, Crown, Mutlilink, GFX stents), stent grafts, vascular or non-vascular grafts, shunts, aneurysm fillers (including GDC, Guglilmi detachable coils), intraluminal paving systems, guide wires, embolic agents (for example, polymeric particles, spheres and liquid embolics), filters (for example, vena cava filters), arteriovenous shunts, artificial heart valves, artificial implants including, but not limited to, prostheses, foreign bodies introduced surgically into the blood vessels, at vascular or non-vascular sites, leads, pacemakers, implantable pulse generators, implantable cardiac defibrillators, cardioverter defibrillators, defibrillators, spinal stimulators, brain stimulators, sacral nerve stimulators, chemical sensors, breast implants, interventional cardiology devices, catheters, amniocentesis and biopsy needles, and the like. Examples of extravascular medical devices and instruments include plastic tubing, dialysis bags or membranes whose surfaces come in contact with the blood stream of a patient, blood oxygenators, blood pumps, blood storage bags, blood collection tubes, blood filters and/or filtration devices, drug pumps, contact lenses, and the like. The term “medical device” also includes bandages or any external device that can be applied directed to the skin.

As used herein, “modulation” means either an increase (stimulation) or a decrease (inhibition) in the expression of a molecule, gene, or activity, for example, the level of nitrosylation of an RyR.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

“Inhibitors”, “activators”, and “modulators” are used to refer to activating, inhibitory, or modulating molecules identified using in vitro and in vivo assays of each of the polynucleotide and polypeptide sequences of these molecules. Inhibitors are compounds that, e.g., bind to, partially or totally block activity, decrease, prevent, delay activation, inactivate, desensitize, or down regulate the activity or expression of, for example, nitric oxide synthase, nitric oxide synthase activity, NO levels, RyR nitrosylation, NADPH oxidase, xanthine oxidoreductase, etc., e.g., antagonists. “Activators” are compounds that increase, open, activate, facilitate, enhance activation, sensitize, agonize, or up regulate e.g. NO synthase activity or levels, e.g., agonists. Inhibitors, activators, or modulators also include genetically modified versions of proteins, for example, nitric oxide synthase, nitric oxide synthase activity, NO levels, RyR nitrosylation, NADPH oxidase, xanthine oxidoreductase, etc., versions with altered activity, as well as naturally occurring and synthetic ligands, substrates, antagonists, agonists, antibodies, peptides, cyclic peptides, nucleic acids, antisense molecules, ribozymes, RNAi, small chemical molecules and the like.

By the term “modulate,” it is meant that any of the mentioned activities, are, e.g., increased, enhanced, increased, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

### Compositions

The contraction of striated muscle is initiated when calcium (Ca²⁺) is released from tubules within the muscle cell known as the sarcoplasmic reticulum (SR). Calcium release channels (ryanodine receptors) on the sarcoplasmic reticulum are required for excitation-contraction (EC) coupling. There are three types of ryanodine receptors, all of which are highly-related Ca²⁺ channels: RyR1, RyR2, and RyR3. RyR1 is found predominantly in skeletal muscle as well as other tissues, RyR2 is found predominantly in the heart as well as other tissues, and RyR3 is found in the brain as well as other tissues. The RyR channels are formed by four RyR polypeptides in association with four FK506 binding proteins (FKBPs), specifically FKBP12 (calstabin1) and FKBP12.6 (calstabin2). Calstabin1 binds to RyR1, calstabin2 binds to RyR2, and calstabin1 binds to RyR3. The FKBP proteins (calstabin1 and calstabin2) bind to the RyR channel (one molecule per RyR subunit), stabilize RyR-channel functioning, and facilitate coupled gating between neighboring RyR channels, preventing abnormal activation of the channel during the channel's closed state. The RyR2 receptor is a tetramer comprised of four 565,000 dalton RyR2 polypeptides and four 12,000 dalton FK-506 binding proteins. The latter are regulatory subunits that stabilize RyR channel function and facilitate coupled gating between neighboring RyR channels which are packed into dense arrays in specialized regions of the sarcoplasmic reticulum that release intracellular stores of Ca²⁺ triggering muscle contraction.

Altered Ca²⁺ homeostasis is a salient feature of heart disease, where the calcium channel ryanodine receptor (RyR) plays a major role. Without wishing to be bound by theory, neuronal nitric oxide synthase (NOS1) regulates the cardiac RyR via S-nitrosylation. The results presented here regarding whether NOS1 deficiency impairs RyR S-nitrosylation, leading to altered Ca²⁺ homeostasis evidence that the theory is correct. In brief, diastolic Ca²⁺ levels were elevated in NOS1 and NOS1/NOS3^{-/-} but not NOS3−/− myocytes compared to wild-type (Wt), indicating diastolic Ca²⁺ leakage. This leak was increased in NOS1^{-/-} and NOS1/NOS3^{-/-} myocytes but not in NOS3^{-/-} cells compared to Wt. NOS1^{-/-} and NOS1/NOS3^{-/-} but not NOS3^{-/-} myocytes exhibited delayed after-depolarizations (DADS). Whereas, the stoichiometry and binding of FKBP12.6 to RyR and RyR phosphorylation were not altered in NOS1^{-/-} hearts, RyR2 S-nitrosylation was near absent. Together these findings, demonstrate that NOS1^{-/-} deficiency causes RyR2 hypo-nitrosylation leading to diastolic Ca²⁺ leak, and a pro-arrhythmic phenotype. NOS1 dysregulation may be a proximate cause of key phenotypes associated with heart disease.

Accordingly, the treatment of cardiac arrhythmia is taken to be the overcoming of the nitric oxide synthase deficiency by, for example, providing treatment such as use of an NO donor agent, nitric oxide enhancer agent, or functional nitric oxide gene which expresses nitric oxide under conditions sufficient to maintain, or return, the regular action of the heart. Cardiac arrhythmia may occur following a disease condition or result from trauma or therapy and the present invention is not limited to any one or more causes of cardiac arrhythmia. Typically, cardiac arrhythmia will occur following myocardial infarction, as a consequence of reperfusion following cardio-pulmonary by-pass surgery, and in patients having unstable angina.

“Nitric oxide enhancing” refers to compounds and functional groups which, under physiological conditions can increase endogenous nitric oxide. Nitric oxide enhancing compounds include, but are not limited to, nitric oxide releasing compounds, nitric oxide donating compounds, nitric oxide donors, radical scavenging compounds and/or reactive oxygen species scavenger compounds. In one embodiment the radical scavenging compound contains a nitroxide group. Nitroxide groups refer to compounds that have the ability to mimic superoxide dimutase and catalase and act as radical scavengers, or react with superoxide or other reactive oxygen species via a stable aminoxyl radical i.e. N-oxide.

“Nitric oxide adduct” or “NO adduct” refers to compounds and functional groups which, under physiological conditions, can donate, release and/or directly or indirectly transfer any of the three redox forms of nitrogen monoxide (NO⁺, NO⁻, NO), such that the biological activity of the nitrogen monoxide species is expressed at the intended site of action.

“Nitric oxide releasing” or “nitric oxide donating” refers to methods of donating, releasing and/or directly or indirectly transferring any of the three redox forms of nitrogen monoxide (NO⁺, NO—, NO), such that the biological activity of the nitrogen monoxide species is expressed at the intended site of action.

“Nitric oxide donor” or “NO donor” refers to compounds that donate, release and/or directly or indirectly transfer a nitrogen monoxide species, and/or stimulate the endogenous production of nitric oxide in vivo and/or elevate endogenous levels of nitric oxide in vivo and/or are oxidized to produce nitric oxide and/or are substrates for nitric oxide synthase. e.g. RyR and/or cytochrome P450. “NO donor” also includes compounds that are precursors of L-arginine, inhibitors of the enzyme arginase and nitric oxide mediators. Heterocyclic nitric oxide donor” refers to a trisubstituted 5-membered ring comprising two or three nitrogen atoms and at least one oxygen atom. The heterocyclic nitric oxide donor is capable of donating and/or releasing a nitrogen monoxide species upon decomposition of the heterocyclic ring. Exemplary heterocyclic nitric oxide donors include oxatriazole-5-ones, oxatriazole-5-imines, sydnonimines, furoxans, and the like.

Nitrogen monoxide can exist in three forms: NO⁻ (nitroxyl), NO (nitric oxide) and NO⁺ (nitrosonium). NO is a highly reactive short-lived species that is potentially toxic to cells. In contrast to the nitric oxide radical (NO), nitrosonium (NO⁺) does not react with O₂ or O₂⁻ species, and functionalities capable of transferring and/or releasing NO⁺ and NO⁻ are also resistant to decomposition in the presence of many redox metals. Consequently, administration of charged NO equivalents (positive and/or negative) does not result in the generation of toxic by-products or the elimination of the active NO group. Nitric oxides encompass uncharged nitric oxide (NO) and charged nitrogen monoxide species, preferably charged nitrogen monoxide species, such as nitrosonium ion (NO⁺) and nitroxyl ion (NO⁻). The reactive form of nitric oxide can be provided by gaseous nitric oxide. The nitrogen monoxide releasing, delivering or transferring compounds have the structure F⁻NO, wherein F is a nitrogen monoxide releasing, delivering or transferring group, and include any and all such compounds which provide nitrogen monoxide to its intended site of action in a form active for its intended purpose. The term “NO adducts” encompasses any nitrogen monoxide releasing, delivering or transferring compounds, including, for example, S-nitrosothiols, nitrites, nitrates, S-nitrothiols, sydnonimines, 2-hydroxy-2-nitrosohydrazines, (NONOates), (E)-alkyl-2-((E)-hydroxyimino)-5-nitro-3-hexeneamide (FK-409), (E)-alkyl-2-((E)-hydroxyimino)-5-nitro-3-hexeneamines, N-((2Z,3E)-4-ethyl-2-(hydroxyimino)-6-methyl-5-nitro-3-heptenyl)-3-pyridi-necarboxamide (FR 146801), N-nitrosoamines, N-hydroxyl nitrosamines, nitrosimines, diazetine dioxides, oxatriazole 5-imines, oximes, hydroxylamines, N-hydroxyguanidines, hydroxyureas, benzofuroxanes, furoxans as well as substrates for the endogenous enzymes which synthesize nitric oxide.

Suitable NONOates include, but are not limited to, (Z)-1-(N-methyl-N-(6-(N-methyl-ammoniohexyl)amino))diazen-1-ium-1,2-diola-te (“MAHMA/NO”), (Z)-1-(N-(3-ammoniopropyl)-N-(n-propyl)amino)diazen-1-ium-1,2-diolate (“PAPA/NO”), (Z)-1-(N-(3-aminopropyl)-N-(4-(3-aminopropylammonio)butyl)-amino)diazen-1-ium-1,2-diolate (spermine NONOate or “SPER/NO”) and sodium(Z)-(N,N-diethylamino)diazenium-1,2-diolate (diethylamine NONOate or “DEA/NO”) and derivatives thereof. NONOates are also described in U.S. Pat. Nos. 6,232,336, 5,910,316 and 5,650,447, the disclosures of which are incorporated herein by reference in their entirety. The NO adducts can be mono-nitrosylated, poly-nitrosylated, mono-nitrosated and/or poly-nitrosated at a variety of naturally susceptible or artificially provided binding sites for biologically active forms of nitrogen monoxide.

Suitable furoxanes include, but are not limited to, CAS 1609, C93-4759, C92-4678, S35b, CHF 2206, CHF 2363, and the like.

Suitable sydnonimines include, but are not limited to, molsidomine(N-ethoxycarbonyl-3-morpholinosydnonimine), SIN-1 (3-morpholinosydnonimine) CAS 936 (3-(cis-2,6-dimethylpiperidino)-N-(4-methoxybenzoyl)-sydnonimine, pirsidomine), C87-3754 (3-(cis-2,6-dimethylpiperidino)sydnonimine, linsidomine, C4144 (3-(3,3-dimethyl-1,4-thiazane-4-yl)sydnonimine hydrochloride), C89-4095 (3-(3,3-dimethyl-1,1-dioxo-1,4-thiazane-4-yl)sydnonimine hydrochloride, and the like.

Suitable oximes, include, but are not limited to, NOR-1, NOR-3, NOR-4, and the like.

One group of NO adducts is the S-nitrosothiols, which are compounds that include at least one —S—NO group. These compounds include S-nitroso-polypeptides (the term “polypeptide” includes proteins and polyamino acids that do not possess an ascertained biological function, and derivatives thereof); S-nitrosylated amino acids (including natural and synthetic amino acids and their stereoisomers and racemic mixtures and derivatives thereof); S-nitrosylated sugars; S-nitrosylated, modified and unmodified, oligonucleotides (preferably of at least 5, and more preferably 5-200 nucleotides); straight or branched, saturated or unsaturated, aliphatic or aromatic, substituted or unsubstituted S-nitrosylated hydrocarbons; and S-nitroso heterocyclic compounds. S-nitrosothiols and methods for preparing them are described in U.S. Pat. Nos. 5,380,758 and 5,703,073; WO 97/27749; WO 98/19672; and Oae et al, Org. Prep. Proc. Int., 15(3):165-198 (1983), the disclosures of each of which are incorporated by reference herein in their entirety.

Another embodiment of the invention is S-nitroso amino acids where the nitroso group is linked to a sulfur group of a sulfur-containing amino acid or derivative thereof. Such compounds include, for example, S-nitroso-N-acetylcysteine, S-nitroso-captopril, S-nitroso-N-acetylpenicillamine, S-nitroso-homocysteine, S-nitroso-cysteine, S-nitroso-glutathione, S-nitroso-cysteinyl-glycine, and the like.

Other examples of suitable S-nitrosothiols include: (i) HS(C(Rₑ)(R_{f}))ₘSNO; (ii) ONS(C(Rₑ)(R_{f}))ₘRₑ; or (iii) H₂N—CH(CO₂H)—(CH₂)ₙ, —C(O)NH—CH(CH₂SNO)—C(O)NH—CH₂—CO₂H, wherein m is an integer from 2 to 20; Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, or Rₑ and R_{f} taken together with the carbons to which they are attached form a carbonyl, a methanthial, a heterocyclic ring, a cycloalkyl group, an aryl group, an oxime, a hydrazone, a bridged cycloalkyl group.

Another group of NO adducts for use in the invention, where the NO adduct is a compound that donates, transfers or releases nitric oxide, include compounds comprising at least one ON—O— or ON—N— group. The compounds that include at least one ON—O— or ON—N— group are preferably ON—O— or ON—N— polypeptides (the term “polypeptide” includes proteins and polyamino acids, and derivatives thereof); ON—O— or ON—N— amino acids (including natural and synthetic amino acids and their stereoisomers and racemic mixtures); ON—O— or ON—N— sugars; ON—O— or —ON—N— modified or unmodified oligonucleotides (comprising at least 5 nucleotides, preferably 5-200 nucleotides); ON—O— or ON—N— straight or branched, saturated or unsaturated, aliphatic or aromatic, substituted or unsubstituted hydrocarbons; and ON—O—, ON—N— or ON—C— heterocyclic compounds. Examples of compounds comprising at least one ON—O— or ON—N— group include butyl nitrite, isobutyl nitrite, tert-butyl nitrite, amyl nitrite, isoamyl nitrite, N-nitrosamines, N-nitrosamides, N-nitrosourea, N-nitrosoguanidines, N-nitrosocarbamates, N-acyl-N-nitroso compounds (such as, N-methyl-N-nitrosourea); N-hydroxy-N-nitrosamines, cupferron, alanosine, dopastin, 1,3-disubstitued nitrosiminobenzimidazoles, 1,3,4-thiadiazole-2-nitrosimines, benzothiazole-2(3H)-nitrosimines, thiazole-2-nitrosimines, oligonitroso sydnonimines, 3-alkyl-N-nitroso-sydnonimines, 2H-1,3,4-thiadiazine nitrosimines.

Another group of NO adducts for use in the invention include nitrates that donate, transfer or release nitric oxide, such as compounds comprising at least one O₂N—O—, O₂N—N— or O₂N—S— group. Among these compounds are O₂N—O—, O₂N—N— or O₂N—S— polypeptides (the term “polypeptide” includes proteins and also polyamino acids that do not possess an ascertained biological function, and derivatives thereof); O₂N—O—, O₂N—N— or O₂N—S— amino acids (including natural and synthetic amino acids and their stereoisomers and racemic mixtures); O₂N—O—, O₂N—N— or O₂N—S— sugars; O₂N—O—, O₂N—N— or O₂N—S— modified and unmodified oligonucleotides (comprising at least 5 nucleotides, preferably 5-200 nucleotides); O₂N—O—, O₂N—N— or O₂N—S— straight or branched, saturated or unsaturated, aliphatic or aromatic, substituted or unsubstituted hydrocarbons; and O₂N—O—, O₂N—N— or O₂N—S— heterocyclic compounds. Examples of compounds comprising at least one O₂N—O—, O₂N—N— or O₂N—S— group include isosorbide dinitrate, isosorbide mononitrate, clonitrate, erythrityl tetranitrate, mannitol hexanitrate, nitroglycerin, pentaerythritoltetranitrate, pentrinitrol, propatylnitrate and organic nitrates with a sulfhydryl-containing amino acid such as, for example SPM 3672, SPM 4757, SPM 5185, SPM 5186 and those disclosed in U.S. Pat. Nos. 5,284,872, 5,428,061, 5,661,129, 5,807,847 and 5,883,122 and in WO 97/46521, WO 00/54756 and in WO 03/013432, the disclosures of each of which are incorporated by reference herein in their entirety.

Another group of NO adducts are N-oxo-N-nitrosoamines that donate, transfer or release nitric oxide and are represented by the formula: R^{1″}R^{2″}N—N(O-M₁⁺)-NO, where R^{1″} and R^{2″} are each independently a polypeptide, an amino acid, a sugar, a modified or unmodified oligonucleotide, a straight or branched, saturated or unsaturated, aliphatic or aromatic, substituted or unsubstituted hydrocarbon, or a heterocyclic group, and where M₁⁺ is an organic or inorganic cation, such, as for example, an alkyl substituted ammonium cation or a Group I metal cation.

Compounds that stimulate endogenous NO and/or are substrates for nitric oxide synthase include, for example, L-arginine, L-homoarginine, and N-hydroxy-L-arginine, N-hydroxy-L-homoarginine, N-hydroxydebrisoquine, N-hydroxypentamidine including their nitrosated and/or nitrosylated analogs (e.g., nitrosated L-arginine, nitrosylated L-arginine, nitrosated N-hydroxy-L-arginine, nitrosylated N-hydroxy-L-arginine, nitrosated and nitrosylated L-homoarginine), N-hydroxyguanidine compounds, amidoxime, ketoximes, aldoxime compounds, that can be oxidized in vivo to produce nitric oxide. Substrates for nitric oxide synthase, cytokines, adenosin, bradykinin, calreticulin, bisacodyl, and phenolphthalein.

Nitric oxide enhancing compounds that can increase endogenous nitric oxide, include for example, nitroxide containing compounds, include, but are not limited to, substituted 2,2,6,6-tetramethyl-1-piperidinyloxy compounds, substituted 2,2,5,5-tetramethyl-3-pyrroline-1-oxyl compounds, substituted 2,2,5,5-tetramethyl-1-pyrrolidinyloxy compounds, substituted 1,1,3,3-tetramethylisoindolin-2-yloxyl compounds, substituted 2,2,4,4-tetramethyl-1-oxazolidinyl-3-oxyl compounds, substituted 3-imidazolin-1-yloxy, 2,2,5,5-tetramethyl-3-imidazolin-1-yloxyl compounds, OT-551, 4-hydroxy-2,2,6,6-tetramethyl-1-piperidinyloxy (tempol), and the like. Suitable substituents, include, but are not limited to, aminomethyl, benzoyl, 2-bromoacetamido, 2-(2-(2-bromoacetamido)ethoxy)ethylcarbamoyl, carbamoyl, carboxy, cyano, 5-(dimethylamino)-1-naphthalenesulfonamido, ethoxyfluorophosphinyloxy, ethyl, 5-fluoro-2,4-dinitroanilino, hydroxy, 2-iodoacetamido, isothiocyanato, isothiocyanatomethyl, methyl, maleimido, maleimidoethyl, 2-(2-maleimidoethoxy)ethylcarbamoyl, maleimidomethyl, maleimido, oxo, phosphonooxy, and the like.

The compounds and compositions of the invention may be used in conjunction with other therapeutic agents for co-therapies, partially or completely, in place of other therapeutic agents, such as, for example, thrombolytic agents, antimicrobial compounds, antiproliferative agents, antisecretory agents, anti-cancer chemotherapeutic agents, steroids, immunosuppressive agents, radiotherapeutic agents, heavy metals functioning as a radiopaque agent, biologic agents, aldosterone antagonists, alpha-adrenergic receptor antagonists, angiotensin II antagonists, angiotensin-converting enzyme (ACE) inhibitors, antidiabetic compounds, anti-hyperlipidemic compounds, antioxidants, antithrombotic and vasodilator compounds, .beta.-adrenergic antagonists, calcium channel blockers, endothelin antagonists, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, and combinations of two or more thereof. The therapeutic agent may optionally be nitrosated and/or nitrosylated and/or contain at least one heterocyclic nitric oxide donor group and/or at least one nitroxide group.

In some embodiments the antioxidants are apocynin, hydralazine compounds and superoxide dimutase mimetics. Suitable hydralazine compounds include, but are not limited to, compounds having the formula:

R₁—N(R₄)—N(R₃)(R₂)

wherein R₁ and R₂ are each independently a hydrogen, an alkyl, an ester or a heterocyclic ring, wherein alkyl, ester and heterocyclic rind are as defined herein; R₃ and R₄ are each independently a lone pair of electrons or a hydrogen, with the proviso that at least one of R.sub.1, R.sub.2, R.sub.3 and R.sub.4 is not a hydrogen. Exemplary hydralazine compounds include budralazine, cadralazine, dihydralazine, endralazine, hydralazine, pildralazine, todralazine, and the like. Suitable hydralazine compounds are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file registry.

In some embodiments the hydralazine compound is hydralazine or a pharmaceutically acceptable salt thereof such as hydralazine hydrochloride. In other embodiments the hydralazine is administered as hydralazine hydrochloride in an amount of about 10 milligrams to about 300 milligrams as a single dose or as multiple doses per day.

The subject of the present invention are in vitro and in vivo systems, including, without limitation, isolated or cultured cells or tissues, non-cell in vitro assay systems and an animal (e.g., an amphibian, a bird, a fish, a mammal, a marsupial, a human, a domestic animal (such as a cat, dog, monkey, mouse or rat) or a commercial animal (such as a cow or pig)).

The cells of a subject include striated muscle cells. A striated muscle is a muscle in which the repeating units (sarcomeres) of the contractile myofibrils are arranged in registry throughout the cell, resulting in transverse or oblique striations that are observed at the level of a light microscope. Examples of striated muscle cells include, without limitation, voluntary (skeletal) muscle cells and cardiac muscle cells. In one embodiment, the cell used in the method of the present invention is a human cardiac muscle cell. As used herein, the term “cardiac muscle cell” includes cardiac muscle fibers, such as those found in the myocardium of the heart. Cardiac muscle fibers are composed of chains of contiguous heart-muscle cells, or cardiomyocytes, joined end to end at intercalated disks. These disks possess two kinds of cell junctions: expanded desmosomes extending along their transverse portions, and gap junctions, the largest of which lie along their longitudinal portions.

In a preferred embodiment, a method of treating cardiac arrhythmias in a subject or patient comprises administering to a patient an agent comprising an NO donor, NO enhancer, or NO adduct in a pharmaceutical composition, wherein the agent modulates ryanodine receptor (RyR) S-nitrosylation to normal levels as compared to a normal subject; and, treating cardiac arrhythmias Administration of the NO donor comprising agent stabilizes the diastolic to calcium (Ca²⁺) levels to normal levels as compared to a normal subject and prevents the leakage of calcium. Modulating and preventing this leak treating with compounds that modulate or inhibit this leak prevents sudden cardiac death.

In another preferred embodiment, treatment of arrhythmia comprises administration of a nucleic acid expressing functional nitric oxide synthase (NO), proteins or peptides thereof, and obtaining ryanodine receptor (RyR) S-nitrosylation normal levels as compared to a normal subject. Administration of the NO gene, protein, peptide and any mutants or modified products thereof, stabilize the diastolic to calcium (Ca²⁺) levels to normal levels as compared to a normal subject and prevents the leakage of calcium.

In another preferred embodiment, a candidate agent modulates the nitrosylation of the ryanodine receptor.

Accordingly, the present invention further provides a method for assaying the effects of the compounds in preventing disorders and diseases associated with the RyR receptors. The method comprises the steps of: (a) obtaining or generating a culture of cells containing RyR; (b) contacting the cells with one or more of the candidate compounds; (c) exposing the cells to one or more conditions known to modulate nitrosylation of RyR in cells; and (d) determining if the one or more compounds modulates the level of RyR-nitrosylation in the cells. As used herein, a cell “containing RyR” is a cell in which RyR, including RyR1, RyR2, and RyR3, or a derivative or homologue thereof, is naturally expressed or naturally occurs. Preferably, the RyR is RyR2. The compounds could also modulate levels of levels of NOS which would affect the S-nitrosylation of RyR2.

In the method of the present invention, cells are contacted with one or more of the compounds by any of the standard methods of effecting contact between drugs/agents and cells, including any modes of introduction and administration described herein. The level of nitrosylation of RyR in the cell is measured or detected by known procedures, including any of the methods, molecular procedures and assays known to one of skill in the art or described herein. In one embodiment of the present invention, the one or more compounds of Formula I limits or prevents a decrease in the level of nitrosylation of RyR and/or increase the activity of NOS.

In another preferred embodiment, the compounds are evaluated for effect on EC coupling and contractility in cells, particularly cardiac muscle cells, and therefore, usefulness for preventing or treating cardiac disorders or cardiac death.

Accordingly, the method of the present invention further comprises the steps of contacting one or more compounds with a culture of cells containing RyR; and determining if the one or more compounds has an effect on an RyR-associated biological event in the cells. As used herein, an “RyR-associated biological event” includes a biochemical or physiological process in which RyR levels or activity have been implicated. As disclosed herein, examples of RyR-associated biological events include, without limitation, EC coupling and contractility in cardiac muscle cells. According to this method of the present invention, the one or more compounds are contacted with one or more cells (such as cardiac muscle cells) in vitro. For example, a culture of the cells is incubated with a preparation containing the one or more compounds. The compounds' effect on a RyR-associated biological event then is assessed by any biological assays or methods known in the art, including immunoblotting, single-channel recordings and any others disclosed herein.

NO/Redox disequilibrium: Heart failure is characterized by impaired excitation-contraction coupling: for instance, increased contractile force upon increasing frequency of stimulation (Force-Frequency relationship, FFR) is compromised in failing hearts, giving rise to a blunted or negative FFR. Defects in Ca²⁺ handling have been described as responsible for this phenomenon. Without wishing to be bound by theory, disruption in signaling pathways due to oxidative stress can be intensified by nitric oxide (NO) deficiency—a situation of nitroso-redox disequilibrium.

Neuronal nitric oxide synthase (NOS1)-mediated NO production plays an important role in excitation-contraction (E-C) coupling, regulation of Ca²⁺ cycling and inhibition of XOR. As a result, deficiency of NOS1 impairs NO/redox equilibrium in the heart, and causes depressed β-adrenergic and force-frequency responses, thus providing a model of the nitroso-redox imbalance observed in the failing heart. In this sense, NO and hydralazine are proposed to exert clinically beneficial effects in HF by restoring the nitroso-redox equilibrium, possibly by acting at the level of XOR and NADPH oxidase (NOX).

The mechanical contraction of the heart is initiated by the spread of an electrical wavefront through the cardiac tissue. This cardiac excitation-contraction coupling is closely linked to the regulation of calcium both outside and inside the myocardial cell. Electrical depolarization of cardiac myocyte results in a small amount of calcium entry into the myocyte through the L-type calcium channels. This small calcium influx causes a calcium induced calcium release from the sarcoplasmic reticulum (SR), an internal cellular structure that stores calcium. The SR released calcium binds with the myocyte actin and myocin, leading to mechanical cell shortening (contraction). The calcium is then sequestered back into the SR, resulting in removal from the actin and myocin and relaxation of the myocyte. Electrical therapies such as post extrasystolic potentiation (PESP) and nonexcitatory electrical stimulation are thought to interact with the cardiac myocyte calcium handling by enhancing SR calcium uptake and L-type calcium influx, respectively.

In a preferred embodiment, organic nitrates and hydralazine improve NO bioavailability and reactivity, ameliorate NO/redox disequilibrium, and restore impaired cardiac excitation-contraction (E-C) coupling.

In another preferred embodiment, candidate therapeutic compounds are identified which result in NO bioavailability and reactivity, ameliorate NO/redox disequilibrium, and restore E-C coupling.

In another preferred embodiment, a candidate therapeutic agent modulates NADPH oxidase and/or xanthine oxidoreductase activity.

In preferred embodiments, the effects of each candidate agent or drug can be further characterized either alone or in combination on ROS production by xanthine oxidoreductase (XOR) and NADPH oxidase (NOX), cardiac excitation-contraction (E-C) coupling, and Ca²⁺ cycling in vitro using, for example, a murine NOS1 knockout as a model of nitroso-redox disequilibrium.

In a preferred embodiment, a candidate agent inhibits NADPH oxidase (NOX) activity, and/or scavenges superoxide and peroxynitrite production.

In another preferred embodiment, a candidate agent inhibits xanthine oxidoreductase (XOR) enzymatic activity and preferably, scavenges superoxide.

In another preferred embodiment, a candidate agent inhibits NADPH oxidase (NOX) activity and xanthine oxidoreductase (XOR) activity. For example, hydralazine and nitroglycerin normalize cardiac E-C coupling impaired by NO/redox disequilibrium by decreasing the amount of ROS produced by XOR and NADPH oxidase and by decreasing diastolic Ca²⁺ leak from the ryanodine receptor.

In another preferred embodiment, a method of diagnosing a patient having nitric oxide deficiency comprises obtaining a sample from a patient; screening the sample for detection of nitric oxide or nitric oxide synthase; determining the levels of nitric oxide in the sample as compared to a normal control.

The levels of nitric oxide in a sample from a patient can be by any means known in the art. This includes commercially available kits, electrochemically, e.g. direct detection of endogenous nitric oxide. The system is comprised from an Fe(II)-dithiocarbamate complex in which TEMPOL molecule coordinates to the centered iron through a redox interaction. Nitric oxide binds to the iron to liberate the TEMPOL (Katayama Y., Chemistry Letters vol. 10, pages: 1152-1153, 2000). Other methods include, for example, immunoassays, Western blotting, PCR, hybridization assays, nitric oxide detection assays, electrochemical assays, fluorometric and colorimetric assays, or electroluminescent assays. Nitric oxide synthase can be detected by one or more methods comprising blotting, hybridization assays, quantification assays, etc.

In a preferred embodiment, the step of detecting and determining levels of nitric oxide or nitric oxide synthase comprises measuring S-nitrosylation of a ryanodine receptor as compared to a control. In the examples section which follows, methods for detecting hyponitrosylation are described in detail.

In another preferred embodiment, a method of diagnosing a patient suffering from a disorder associated with nitric oxide deficiency comprising obtaining a sample from a patient; and, assessing levels of nitrosylation of a ryanodine receptor as compared to a normal control. Preferably, the patient suffering from a disorder associated with nitric oxide deficiency, more preferably, the disorder is associated with nitric oxide deficiency resulting in decreased levels of ryanodine receptor nitrosylation, such as for example, cardiac disorders and cardiac diseases.

In another preferred embodiment, the prognosis for recovery from heart diseases or disorders comprises measuring the levels of RyR nitrosylation and comparing said levels to a normal healthy control. Decreased amounts evidence a poor prognosis.

In another preferred embodiment, a patient at risk of developing a cardiac disease, cardiac disorder, comprises assessing the nitric oxide levels, nitric oxide synthase levels or activity, or S-nitrosylation of the ryanodine receptor.

### Candidate Therapeutic Agents

Embodiments of the invention encompass methods of screening compounds to identify those agent inhibits NADPH oxidase (NOX) activity and xanthine oxidoreductase (XOR) activity. Compounds will also be screened for the nitrosylation of ryanodine receptors. Screening assays for drug candidates will include assays amenable to high-throughput screening of chemical libraries, making them particularly suitable for identifying small molecule drug candidates.

The methods include administering the compound to a model of the condition, e.g., contacting a cell (in vitro) model with the compound, or administering the compound to an animal model of the condition. The model is then evaluated for an effect of the candidate compound on at least one of NOX activity, XOR activity, E-C coupling and nitrosylation of ryanodine receptors.

The test compounds utilized in the assays and methods described herein can be, inter alia, nucleic acids, small molecules, organic or inorganic compounds, antibodies or antigen-binding fragments thereof, polynucleotides, peptides, or polypeptides. For example, polypeptide variants including truncation mutants, deletion mutants, and point mutants; nucleic acids including sense, antisense, aptamers, and small inhibitory RNAs (siRNAs) including short hairpin RNAs (shRNAs) and ribozymes) can be used as test compounds in the methods described herein.

A candidate compound that has been screened, e.g., in an in vitro or in vivo model, and determined to have a desirable effect on one or more modulatory activities associated with NOX, XOR, E-C coupling and nitrosylation of ryanodine receptors, can be considered a candidate therapeutic agent. Candidate therapeutic agents, once screened in a clinical setting, are therapeutic agents, and both types of agents can be optionally optimized (e.g., by derivatization), and formulated with pharmaceutically acceptable excipients or carriers to form pharmaceutical compositions.

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

In some embodiments, the compounds are optimized to improve their therapeutic index, i.e., increase therapeutic efficacy and/or decrease unwanted side effects. Thus, in some embodiments, the methods described herein include optimizing the test or candidate compound. In some embodiments, the methods include formulating a therapeutic composition including a test or candidate compound (e.g., an optimized compound) and a pharmaceutically acceptable carrier. In some embodiments, the compounds are optimized by derivatization using methods known in the art.

In one embodiment, screening comprises contacting the core molecules in the assays described herein with a diverse library of member compounds. The compounds or “candidate therapeutic agents” can be any organic, inorganic, small molecule, protein, antibody, aptamer, nucleic acid molecule, or synthetic compound.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods, can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with β-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

### Pharmaceutical Composition

The compounds or candidate agents of the invention are formulated into pharmaceutical compositions for administration to human subjects in a biologically compatible form suitable for administration in vivo. According to another aspect, the present invention provides a pharmaceutical composition comprising compounds in admixture with a pharmaceutically acceptable diluent and/or carrier. The pharmaceutically-acceptable carrier must be “acceptable” in the sense of being compatible with the other ingredients of the composition and not deleterious to the recipient thereof. The pharmaceutically-acceptable carrier employed herein is selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. If necessary, pharmaceutical additives, such as antioxidants, aromatics, colorants, flavor-improving agents, preservatives, and sweeteners, are also added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others.

The pharmaceutical formulations of the present invention are prepared by methods well-known in the pharmaceutical arts. For example, the compounds of are brought into association with a carrier and/or diluent, as a suspension or solution. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

The compounds are administered to a subject by contacting target cells (e.g., cardiac muscle cells) in vivo in the subject with the compounds. The compounds are contacted with (e.g., introduced into) cells of the subject using known techniques utilized for the introduction and administration of proteins, nucleic acids and other drugs. Examples of methods for contacting the cells with (i.e., treating the cells with) the compounds include, without limitation, absorption, electroporation, immersion, injection, introduction, liposome delivery, transfection, transfusion, vectors and other drug-delivery vehicles and methods. When the target cells are localized to a particular portion of a subject, the compounds can be, if desired, introduced directly to the cells, by injection or by some other means (e.g., by introducing the compounds into the blood or another body fluid). The target cells are contained in tissue of a subject and are detected by standard detection methods readily determined from the known art, examples of which include, without limitation, immunological techniques (e.g., immunohistochemical staining), fluorescence imaging techniques, and microscopic techniques.

Additionally, the compounds are administered to a human or animal subject by known procedures including, without limitation, oral administration, sublingual or buccal administration, parenteral administration, transdermal administration, via inhalation or intranasally, vaginally, rectally, and intramuscularly. The compounds are administered parenterally, by epifascial, intracapsular, intracranial, intracutaneous, intrathecal, intramuscular, intraorbital, intraperitoneal, intraspinal, intrasternal, intravascular, intravenous, parenchymatous, subcutaneous or sublingual injection, or by way of catheter. In one embodiment, the agent is administered to the subject by way of delivery to the subject's muscles including, but not limited to, the subject's cardiac muscles. In an embodiment, the agent is administered to the subject by way of targeted delivery to cardiac muscle cells via a catheter inserted into the subject's heart.

For oral administration, a formulation of the compounds are presented as capsules, tablets, powders, granules or as a suspension or solution. The formulation has conventional additives, such as lactose, mannitol, corn starch or potato starch. The formulation also is presented with binders, such as crystalline cellulose, cellulose derivatives, acacia, corn starch or gelatins. Additionally, the formulation is presented with disintegrators, such as corn starch, potato starch or sodium carboxymethylcellulose. The formulation also is presented with dibasic calcium phosphate anhydrous or sodium starch glycolate. Finally, the formulation is presented with lubricants, such as talc or magnesium stearate.

For parenteral administration (i.e., administration by injection through a route other than the alimentary canal), the compounds are combined with a sterile aqueous solution that is isotonic with the blood of the subject. Such a formulation is prepared by dissolving a solid active ingredient in water containing physiologically-compatible substances, such as sodium chloride, glycine and the like, and having a buffered pH compatible with physiological conditions, so as to produce an aqueous solution, then rendering said solution sterile. The formulation is presented in unit or multi-dose containers, such as sealed ampoules or vials. The formulation is delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual or by way of catheter into the subject's heart.

For transdermal administration, the compounds are combined with skin penetration enhancers, such as propylene glycol, polyethylene glycol, isopropanol, ethanol, oleic acid, N-methylpyrrolidone and the like, which increase the permeability of the skin to the compounds and permit the compounds to penetrate through the skin and into the bloodstream. The compound/enhancer composition also are further combined with a polymeric substance, such as ethylcellulose, hydroxypropyl cellulose, ethylene/vinylacetate, polyvinyl pyrrolidone, and the like, to provide the composition in gel form, which are dissolved in a solvent, such as methylene chloride, evaporated to the desired viscosity and then applied to backing material to provide a patch.

In some embodiments, the composition is in unit dose form such as a tablet, capsule or single-dose vial. Suitable unit doses, i.e., therapeutically effective amounts, can be determined during clinical trials designed appropriately for each of the conditions for which administration of a chosen compound is indicated and will, of course, vary depending on the desired clinical endpoint. The present invention also provides articles of manufacture for treating and preventing disorders, such as cardiac disorders, in a subject. The articles of manufacture comprise a pharmaceutical composition of one or more of the identified compounds. The articles of manufacture are packaged with indications for various disorders that the pharmaceutical compositions are capable of treating and/or preventing. For example, the articles of manufacture comprise a unit dose of a compound disclosed herein that is capable of treating or preventing a muscular disorder, and an indication that the unit dose is capable of treating or preventing a certain disorder, for example an arrhythmia.

In accordance with a method of the present invention, the compounds are administered to the subject (or are contacted with cells of the subject) in an amount effective to modulate nitrosylation of the RyR, which can be mediated by nitric oxide synthase (NOS) or modulate NOS expression or activity in the subject, particularly in cells of the subject.

In another preferred embodiment, the compounds are administered to the subject (or are contacted with cells of the subject) in an amount effective to modulate xanthine oxidoreductase (XOR) and/or NADPH oxidase activity, thereby modulating inter alia, reactive oxygen species (ROS) production.

This amount is readily determined by the skilled artisan, based upon known procedures, including analysis of titration curves established in vivo and methods and assays disclosed herein. A suitable amount of the compounds in the subject ranges from about 5 mg/kg/day to about 20 mg/kg/day, and/or is an amount sufficient to achieve plasma levels ranging from about 300 ng/ml to about 1000 ng/ml.

### Cellular Compositions

In a preferred embodiment, a cell is isolated from a subject, preferably a mammal. The cell comprises a defective nitric oxide synthase gene wherein nitric oxide is decreased as compared to a normal control. The cell can be useful in assays and diagnostic applications in identifying agents which may overcome the defect and produce normal levels of nitric oxide.

In another preferred a cell is nitric oxide deficient. The nitric oxide deficient cell can be contacted with a vector expressing nitric oxide synthase. In another preferred embodiment, the nitric oxide synthase deficient cell produces nitric oxide when contacted with an agent that overcomes a defect in the cells ability to produce nitric oxide.

In another preferred embodiment, a cell comprises a vector comprising a promoter operably linked to a polynucleotide expressing at least one of nitric oxide synthase polynucleotide, ryanodine receptor, NADPH oxidase, xanthine oxidoreductase, variants, mutants and fragments thereof.

In another preferred embodiment, a stem cell is contacted with a a vector comprising a promoter operably linked to a polynucleotide expressing at least one of nitric oxide synthase polynucleotide, ryanodine receptor, NADPH oxidase, xanthine oxidoreductase, variants, mutants and fragments thereof.

In a preferred embodiment, the promoter can be an inducible or tissue specific promoter.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.

Retroviral vectors typically comprise the RNA of a transmissible agent, into which a heterologous sequence encoding a protein of interest is inserted. Typically, the retroviral RNA genome is expressed from a DNA constrict. A common way to insert one segment of DNA into another segment of DNA involves the use of enzymes called restriction enzymes that cleave DNA at specific sites (specific groups of nucleotides) called restriction sites. A “cassette” refers to a DNA segment that can be inserted into a vector or into another piece of DNA at a defined restriction site. Preferably, a cassette is an “expression cassette” in which the DNA is a coding sequence or segment of DNA that codes for an expression product that can be inserted into a vector at defined restriction sites. The cassette restriction sites generally are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of DNA construct is a “plasmid” that generally is a self-contained molecule of double-stranded DNA, usually of bacterial origin, that can readily accept additional (foreign) DNA and which can be readily introduced into a suitable producer cell. A plasmid vector often contains coding DNA and promoter DNA and has one or more restriction sites suitable for inserting foreign DNA. A large number of vectors, including plasmid and fungal vectors, have been described for replication and/or expression in a variety of eukaryotic and prokaryotic hosts. Non-limiting examples include pKK plasmids (Amersham Pharmacia Biotech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes. A “retroviral plasmid vector” means a plasmid which includes all or part of a retroviral genome including 5′ and 3′ retroviral long-term repeat (LTR) sequences, a packaging signal (ψ), and may include one or more polynucleotides encoding a protein(s) or polypeptide(s) of interest, such as a therapeutic agent or a selectable marker. Such retroviral plasmid vectors are described, e.g., in U.S. Pat. No. 5,952,225 which is specifically incorporated herein by reference.

Expression of a peptide, e.g. nitric oxide synthase, fragments thereof, conservative variant thereof, or analog or derivative thereof of the invention may be controlled by promoter/enhancer elements disclosed herein, but these regulatory elements must be functional in the host selected for expression. Promoters which may be used to control gene expression include but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR, see Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamHI, SfiI, XhoI, NotI, NheI, HindIII, NheI, PvuII, and KpnI cloning site, constitutive RSV-LTR promoter, hygromycin selectable marker; Invitrogen), pCEP4 (BamHI, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamHII cloning site, inducible metallothionein IIa gene promoter, hygromycin selectable marker; Invitrogen), pREP8 (BamHI, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSCII (SmaI cloning site, TK- and β-gal selection), pMJ601 (SalI, SmaI, AflI, NarI, BspMII, BamHI, ApaI, NheI, SacII, KpnI, and HindIII cloning site; TK- and β-gal selection), and pTKgptFIS (EcoRI, PsiI, SalI, AccI, HindIII, SbaI, BamHI, and Hpa cloning site, TK or XPRT selection).

The molecules of interest are operably linked to the promoter. A nucleic acid is “operably linked” when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

Vectors are introduced into desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter.

The term “progenitor cell” is used synonymously with “stem cell.” Both terms refer to an undifferentiated cell which is capable of proliferation and giving rise to more progenitor cells having the ability to generate a large number of mother cells that can in turn give rise to differentiated, or differentiable daughter cells.

In a preferred embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors.

In the context of cell ontogeny, the adjective “differentiated” is a relative term. A “differentiated cell” is a cell that has progressed further down the developmental pathway than the cell it is being compared with. Thus, pluripotent embryonic stem cells can differentiate to lineage-restricted precursor cells, such as hematopoietic cells, which are pluripotent for blood cell types; hepatocyte progenitors, which are pluripotent for hepatocytes; and various types of neural progenitors listed above. These in turn can be differentiated further to other types of precursor cells further down the pathway, or to an end-stage differentiated cell, which plays a characteristic role in a certain tissue type, and may or may not retain the capacity to proliferate further. Neurons, astrocytes, and oligodendrocytes are all examples of terminally differentiated cells.

### Uses

The present invention provides a new range of therapeutic treatments for patients with various disorders involving modulation of the RyR receptors, particularly cardiac (RyR2) disorders. However, disorders associated with the other ryanodine receptors can be treated with compounds identified by the methods described herein, and include, for example, skeletal muscular disorders (RyR1), and cognitive (RyR3) disorders.

In one embodiment of the present invention, the subject has not yet developed a disorder, such as cardiac disorders (e.g., exercise-induced cardiac arrhythmia) In another embodiment of the present invention, the subject is in need of treatment for a disorder, including a cardiac disorder.

Various disorders that the compounds treat or prevent include, but are not limited to, cardiac disorders and diseases, skeletal muscular disorders and diseases, cognitive disorders and diseases, malignant hyperthermia, diabetes, and sudden infant death syndrome. Cardiac disorder and diseases include, but are not limited to, irregular heartbeat disorders and diseases; exercise-induced irregular heartbeat disorders and diseases; sudden cardiac death; exercise-induced sudden cardiac death; congestive heart failure; chronic obstructive pulmonary disease; and high blood pressure. Irregular heartbeat disorders and diseases include and exercise-induced irregular heartbeat disorders and diseases include, but are not limited to, atrial and ventricular arrhythmia; atrial and ventricular fibrillation; atrial and ventricular tachyarrhythmia; atrial and ventricular tachycardia; catecholaminergic polymorphic ventricular tachycardia (CPVT); and exercise-induced variants thereof. Skeletal muscular disorder and diseases include, but are not limited to, skeletal muscle fatigue, exercise-induced skeletal muscle fatigue, muscular dystrophy, bladder disorders, and incontinence. Cognitive disorders and diseases include, but are not limited to, Alzheimer's Disease, forms of memory loss, and age-dependent memory loss. One skilled in the art will recognize still other diseases, including but not limited to muscular and cardiac disorders, that the compounds are useful to treat, in accordance with the information provided herein.

The amount of compounds administered to a subject will vary depending upon the particular factors of each case, including the subject's weight, the severity of the subject's condition, and the mode of administration. This amount is readily determined by the skilled artisan, based upon known procedures, including clinical trials, and methods disclosed herein.

The compounds can be used alone, in combination with each other, or in combination with other agents that have cardiovascular activity including, but not limited to, diuretics, anticoagulants, antiplatelet agents, antiarrhythmics, inotropic agents, chronotropic agents, α and β blockers, angiotensin inhibitors and vasodilators. Further, such combinations of the compounds of the present invention and other cardiovascular agents are administered separately or in conjunction. In addition, the administration of one element of the combination is prior to, concurrent to or subsequent to the administration of other agent(s).

In another preferred embodiment, the invention is further directed to one or more compounds identified by the above-described identification method, as well as a pharmaceutical composition comprising the compound and a pharmaceutically acceptable carrier and/or diluent. The compounds are useful for treating or preventing RyR-associated conditions, nitric oxide synthase deficiencies, NADPH oxidase and/or xanthine oxidoreductase modulation. As used herein, an “RyR-associated condition” is a condition, disease, or disorder in which RyR level or activity has been implicated, and includes an RyR-associated biological event. The RyR-associated condition is treated or prevented in the subject by administering to the subject an amount of the compound effective to treat or prevent the RyR-associated condition in the subject. This amount is readily determined by one skilled in the art.

The present invention also provides an in vivo method for assaying the effectiveness of the compounds in preventing disorders and diseases associated with RyR nitrosylation levels , nitric oxide synthase deficiencies, NADPH oxidase and/or xanthine oxidoreductase modulation. The method comprises the steps of: (a) obtaining or generating an animal containing RyR; (b) administering one or more of the compounds to the animal; (c) exposing the animal to one or more conditions known to decrease nitrosylation of RyR in cells; and (d) determining the extent the compound modulates nitrosylation of RyR in the animal. The method further comprises the steps of: (e) administering one or more of the compounds to an animal containing RyR; and (f) determining the extent of the effect of the compound on a RyR-associated biological event in the animal.

The present invention also provides a method of diagnosis of a disease or disorder in a subject, said method comprising: obtaining a cell or tissue sample from the subject; obtaining DNA from the cell or tissue; comparing the DNA from the cell or tissue with a control DNA encoding RyR or nitric oxide synthase to determine whether a mutation is present in the DNA from the cell or tissue, the presence of a mutation indicating a disease or disorder. The mutation is one preventing endogenous nitrosylation of RyR. The disease and disorders include, but are not limited to, cardiac disorders and diseases, including, but are not limited to, irregular heartbeat disorders and diseases; exercise-induced irregular heartbeat disorders and diseases; sudden cardiac death; exercise-induced sudden cardiac death; congestive heart failure; chronic obstructive pulmonary disease; and high blood pressure. Irregular heartbeat disorders and diseases include and exercise-induced irregular heartbeat disorders and diseases include, but are not limited to, atrial and ventricular arrhythmia; atrial and ventricular fibrillation; atrial and ventricular tachyarrhythmia; atrial and ventricular tachycardia; catecholaminergic polymorphic ventricular tachycardia (CPVT); and exercise-induced variants thereof. Skeletal muscular disorder and diseases include, but are not limited to, skeletal muscle fatigue, exercise-induced skeletal muscle fatigue, muscular dystrophy, bladder disorders, and incontinence. Cognitive disorders and diseases include, but are not limited to, Alzheimer's Disease, forms of memory loss, and age-dependent memory loss.

The present invention further provides a method of diagnosis of a cardiac disorder or disease in a subject, said method comprising: obtaining cardiac cells or tissue sample from the subject; determining the nitrosylation levels of RyR, nitric oxide synthase activity, NADPH oxidase activity, and/or xanthine oxidoreductase activity.

In addition to the above-mentioned therapeutic uses, the compounds of the invention are also useful in diagnostic assays, screening assays and as research tools.

### High-Throughput Screening Method

In addition to the compounds disclosed herein, other compounds can be discovered that are capable of modulating nitrosylation of RyR, nitric oxide synthase activity, NADPH oxidase activity (NOX), and/or xanthine oxidase activity (XOR).

In another preferred embodiment, a compound comprises antisense molecules specific for NADPH oxidase or xanthine oxidoreductase. The antisense molecules comprise non-enzymatic oligonucleotide molecules that bind to another oligonucleotide or target nucleic acid e.g. NOX or XOR, for example, RNA, DNA (target) by means of interactions between the oligonucleotides and alters the activity of the target nucleic acids (Eguchi et al., 1991 Annu. Rev. Biochem. 60, 631-652).

An antisense compound is specifically hybridizable when binding of the compound to the target DNA or RNA molecule interferes with the normal function of the target DNA or RNA to cause a loss of utility, and there is a sufficient degree of complementarily to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. In the context of this invention, the term Oligonucleotides refers to an oligomer or polymer of ribonucleic acid (RNA) or deoxyribonucleic acid (DNA) or mimetics thereof.

As used herein, the term “oligonucleotide”, includes linear or circular oligomers of natural and/or modified monomers or linkages, including deoxyribonucleosides, ribonucleosides, substituted and alpha-anomeric forms thereof, peptide nucleic acids (PNA), ed nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like.

The oligonucleotide may be “chimeric”, that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleosides and/or oligonucleotide analogs as described above.

The oligonucleotide can be composed of regions that can be linked in “register”, that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the terms “target nucleic acid” and encompass DNA, RNA (comprising pre-mRNA and mRNA) transcribed from such DNA, and also cDNA derived from such RNA. In a preferred embodiment, the target nucleic acids comprise NADPH oxidase, or xanthine oxidoreductase. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA to be interfered include, for example, replication and transcription. The functions of RNA to be interfered, include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of an encoded product or oligonucleotides.

The above procedures can be fully automated. For example, sampling of sample materials may be accomplished with a plurality of steps, which include withdrawing a sample from a sample container and delivering at least a portion of the withdrawn sample to the assay. Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

In the general case, sampling can be effected manually, in a semi-automatic manner or in an automatic manner. A sample can be withdrawn from a sample container manually, for example, with a pipette or with a syringe-type manual probe, and then manually delivered to a loading port or an injection port of a characterization system. In a semi-automatic protocol, some aspect of the protocol is effected automatically (e.g., delivery), but some other aspect requires manual intervention (e.g., withdrawal of samples from a process control line). Preferably, however, the sample(s) are withdrawn from a sample container and delivered to the characterization system, in a fully automated manner, for example, with an auto-sampler.

In one embodiment, auto-sampling may be done using a microprocessor controlling an automated system (e.g., a robot arm). Preferably, the microprocessor is user-programmable to accommodate libraries of samples having varying arrangements of samples (e.g., square arrays with “n-rows” by “n-columns,” rectangular arrays with “n-rows” by “m-columns,” round arrays, triangular arrays with “r-” by “r-” by “r-” equilateral sides, triangular arrays with “r-base” by “s-” by “s-” isosceles sides, etc., where n, m, r, and s are integers).

Automated sampling of sample materials optionally may be effected with an auto-sampler having a heated injection probe (tip). An example of one such auto sampler is disclosed in U.S. Pat. No. 6,175,409 B1 (incorporated by reference).

According to the present invention, one or more systems, methods or both are used to identify a plurality of sample materials. Though manual or semi-automated systems and methods are possible, preferably an automated system or method is employed. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

Generally, the automated system includes a suitable protocol design and execution software that can be programmed with information such as synthesis, composition, location information or other information related to a library of materials positioned with respect to a substrate. The protocol design and execution software is typically in communication with robot control software for controlling a robot or other automated apparatus or system. The protocol design and execution software is also in communication with data acquisition hardware/software for collecting data from response measuring hardware. Once the data is collected in the database, analytical software may be used to analyze the data, and more specifically, to determine properties of the candidate drugs, or the data may be analyzed manually.

### Kits

In a preferred embodiment, a kit for detecting comprises one or more components for detecting nitric oxide, nitric oxide synthase, or nitrosylation of a ryanodine receptor. The kits may also include buffer and/or excipient solutions (in liquid or frozen form)—or buffer and/or excipient powder preparations to be reconstituted with water. Thus, preferably the kits containing the components, are frozen, lyophilized, pre-diluted, or pre-mixed at such a concentration that the addition of a predetermined amount of heat, of water, or of a solution provided in the kit will result in a formulation of sufficient concentration and pH as to be effective assaying any compound for therapeutic value in the treatment of disease. Preferably, such a kit will also comprise instructions for reconstituting and using the components of the assay. The kit may also comprise two or more component parts for the reconstituted active composition. The above-noted buffers, excipients, and other component parts can be sold separately or together with the kit.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### Examples

The following non-limiting Examples serve to illustrate selected embodiments of the invention. It will be appreciated that variations in proportions and alternatives in elements of the components shown will be apparent to those skilled in the art and are within the scope of embodiments of the present invention.

### Materials and Methods

Animals

Transgenic mice (males and females, 3-6 moths old) with homozygous deletions of NOS1 (Huang, P. L., et al., 1993. Cell 75:1273-1286), NOS3 (Huang, P. L., 1995. Nature 377:239-242), and double NOS1/NOS3 knock-out (Son, H., et al. Cell 87:1015-1023) bred on a C57BL/6 background, that was used as wild type (Jackson Laboratories, Bar Harbor, Me.). All protocols and experimental procedures were approved by the Animal Care and Use Committee of The Johns Hopkins University School of Medicine and the Miller School of Medicine.

Isolation of Myocytes

The hearts were cannulated and perfused trough the aorta with Ca²⁺-free bicarbonate buffer containing (in mM) 120 NaCl, 5.4 KCl, 1.2 MgSO₄, 1.2 NaH₂PO₄, 5.6 glucose, 20 NaHCO₃, 10 2,3-butanedione monoxime (BDM; Sigma), and 5 taurine (Sigma), gassed with 95% O₂-5% CO₂, followed by enzymatic digestion with collagenase type 2 (1 mg/ml; Worthington) and protease type XIV (0.1 mg/ml; Sigma). Ventricular myocytes were obtained by mechanical disruption of digested hearts, filtration, centrifugation, and resuspension in a Tyrode solution containing (in mM) 0.125 mM CaCl₂ 144 NaCl, 1 MgCl₂, 10 HEPES, 5.6 Glucose, 1.2 NaHPO₄, 5 KCl, adjusted to a pH of 7.4 with NaOH. Then, the Ca²⁺ content of the buffer was increased in steps until reaching 1.8 mM.

Myocytes were incubated with 5 μmol/L fura-2/AM (Molecular Probes, Eugene, Oreg.) then transferred to a Lucite chamber on the stage of an inverted microscope (Nikon TE 200), continuously superfused with Tyrode solution. Sarcomere length (SL) and Ca²⁺ transients ([Ca²⁺]ᵢ) were measured in myocytes stimulated at 1, 2, 4, 6 and 8 Hz. All experiments were conducted at 37° C.

Sarcomere length (SL) was recorded with an IonOptix iCCD camera. Change in average SL was determined by fast Fourier transform of the Z-line density trace to the frequency domain, and SL Shortening was calculated as follows: (diastolic SL-systolic SL)/diastolic SL. [Ca²⁺]ᵢ was measured using the Ca²⁺-sensitive dye fura-2 and a dual-excitation spectrofluorometer (IonOptix, Milton, Mass.), alternately excited with a xenon lamp at wavelengths of 365 and 380 nm The emission fluorescence was reflected through a barrier filter (510±15 nm) to a photomultiplier tube. The fura-2 fluorescence ratio, the ratio of the photon live count detected by the excitation at 365 nm compared with 380 nm, represents [Ca²⁺]ᵢ.

Assessment of SR Ca²⁺ Leakage

Calcium leakage was assessed as described by Shannon et al (Shannon, T. R. et al., 2002. Circ. Res. 91:594-600). Ventricular myocytes were paced by field stimulation at the different frequencies in normal Tyrode until cellular Ca²⁺ transients reached a steady state. After the last pulse, the superfusing solution was rapidly switched to 0Na⁺-0Ca²⁺ (Na⁺ replaced by Li⁺) Tyrode with or without 1 mmol/L tetracaine. In the control condition, [Ca²⁺]ᵢ was monitored while 0Na⁺-0Ca²⁺ Tyrode buffer was applied at least 40 seconds, then a rapid pulse of 10 mm/L caffeine was added to cause SR Ca²⁺ release. The difference between the basal and the peak total cytosolic [Ca²⁺]ᵢ in the presence of caffeine is therefore [Ca²⁺]_{T}. In the test condition, 0Na+0Ca²⁺ Tyrode was applied with tetracaine. In this condition, the leakage is blocked and the difference in [Ca²⁺]_{T} between tetracaine and control condition corresponds to diastolic leakage. To calculate [Ca²⁺]_{T} in the SR, the total SR volume was considered 3% of the cell volume and the cytosol volume, 65% of the cell volume.

[Ca²⁺]ᵢ Calibration

The signal of fura 2-AM was measured as a ratio of the emission at 365/380 nm This signal was converted to [Ca²⁺]ᵢ using the method described by Grynkiewicz et al (J. Biol. Chem. (1985) 260:3440-3450), using the function:

[Ca²⁺]ᵢ=K_{d}(F−Fₘᵢₙ)/(Fₘₐₓ−F)

where Kd is the dissociation constant for fura-2 at 350, F is the ratio of the fluorescence at 365/380 of the fluorescence, Fₘᵢₙ is the minimal signal at 0Ca²⁺ conditions and Fₘₐₓ is the value obtained at saturating Ca²⁺ conditions, both using permeabilized cardiomyocytes with ionomycin.

Western Blotting:

Hearts were cannulated and perfused trough the aorta, with Krebs solution to wash blood and then rapidly frozen in liquid nitrogen. Equal amounts of protein (30 μg) were resolved by NuPAGE 3-8% Tris-acetate gels (Invitrogen Life Technologies, Carlsbad, Calif.) to detect RyR2. 12% Bis-Tris gels were used to visualize PLB, FKBP12.6 and calsequestrin. Proteins were transferred to a PVDF membrane (Bio-Rad, Hercules, Calif.) and then blocked overnight by incubation with 5% nonfat dry milk in Tris-buffered saline (TBS). After washing with TBS containing 0.1% Tween 20 (TBS-T buffer), the blots were incubated with primary antibodies, including mouse anti-RyR2 (Affinity Bioreagents, Golden, Colo.) and PLB (Abcam, Cambridge, Mass.), anti-NCX (RDI, Concord, Mass.) rabbit polyclonal anti FKBP12.6 (Abcam) anti calsequestrin (RDI) and anti phospho RyR and phospho Ser16 PLB (Badrilla, UK). RyR was immunoprecipitated using the monoclonal antibody from and protein 0 coupled to sepharose (Pierce, Rockford, Ill.).

Assessment of S-Nitrosylation

For determination of RyR nitrosylation, hearts were treated as above and the biotin switch technique was performed accordingly to Jaifrey et al (Jaffrey, S. R. et al., (2001) Sci. STKE. 2001:L1; Jaffrey, S. R., et al. (2001) Nat. Cell Biol. 3:193-197). Briefly, hearts were homogenized in HEN buffer. Free cysteine residues were blocked with S-methyl methanethiosulfonate. Proteins were precipitated with cold acetone and then resuspended in HEN buffer containing 0.5% SDS and labeled with biotin-HPDP with or without sodium ascorbate. For direct detection of biotinylated RyR2, 60 pg of each sample was loaded in a 3-8% Tris-acetate gel, electrophoretically resolved and followed by immunoblotting with anti-biotin antibody. After stripping the blotting membranes, they were re-probed with anti-RyR2. In order to separate S-nitrosylated RyR2 after labeling, biotinylated proteins were incubated with streptavidin-agarose beads. The bound proteins were eluted with β-mercaptoethanol and resolved electrophoretically in a 3-8% Tris-acetate gel, followed by immunoblotting with anti-RyR2 and anti GAPDH.

Statistical Analysis

Data are expressed as mean±s.e.m. For comparisons of two groups, unpaired two tailed Student's t test was used. For comparison of more than three groups, ANOVA (one or two ways as appropriate) was performed with Bonferroni's as a post-hoc test. For all tests, a P value of less than 0.05 was considered to be significant.

### Example 1

### Deficiency in S-Nitrosylation of the Ryanodine Receptor Increases Sarcoplasmic Reticulum Calcium Leak and Arrhythmogenesis in Cardiomyocytes

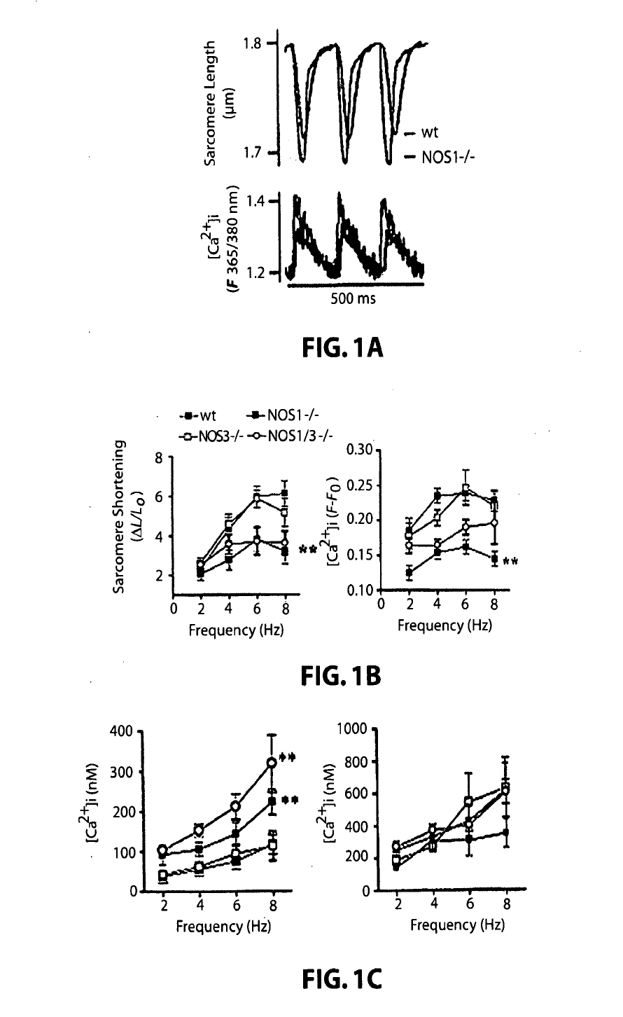

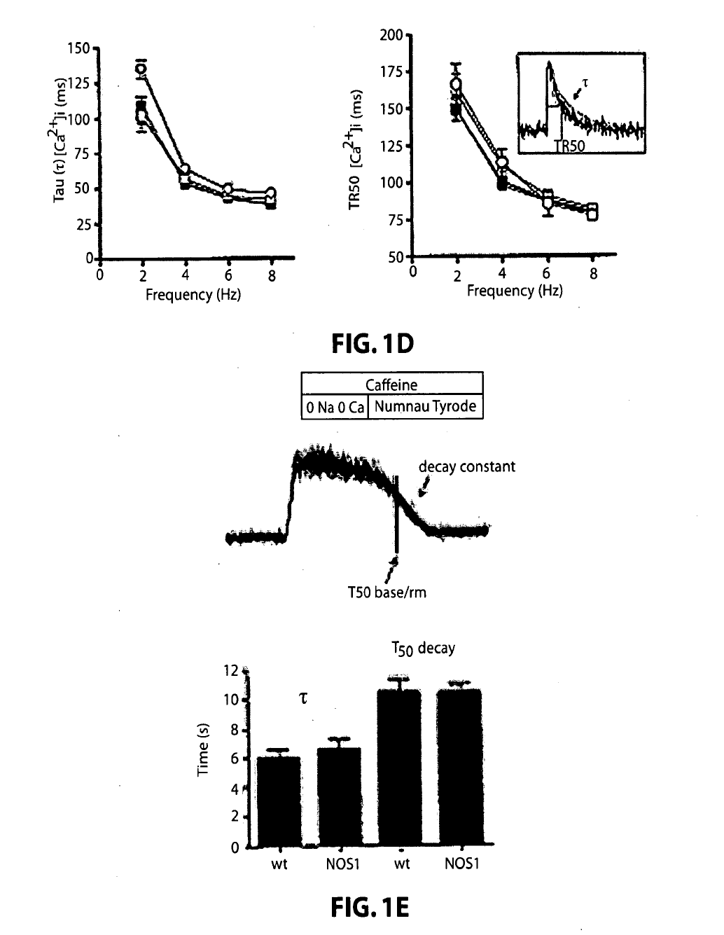

Force-Frequency relationship and diastolic [Ca²⁺]ᵢ: The force frequency relationship is depressed in NOS1^{-/-} mice. Consistent with these previous observations, when field stimulated at 2, 4, 6 and 8 Hz, the degree of sarcomere shortening and the amplitude of calcium transients in cardiomyocytes (FIG. 1B) were significantly reduced in NOS1^{-/-} compared to both wild type and NOS3^{-/-} myocytes.

As reduced Ca²⁺ transients could be due to either RyR dysfunction or impaired Ca²⁺ reuptake into the SR, systolic and diastolic levels of [Ca²⁺]ᵢ were measured in the four strains of mice. It was noted that diastolic [Ca²⁺]ᵢ levels rose over the full range of stimulation frequencies in NOS1^{-/-} and NOS3/NOS1^{-/-} but not NOS3^{-/-} myocytes (FIG. 1C), a finding evidencing diastolic [Ca²⁺]ᵢ leak. Conversely, it was found that parameters of [Ca²⁺]ᵢ reuptake, τ and TR50 (time to achieve 50% of decay), were not different between wild type, NOS1^{-/-}, NOS3^{-/-} and NOS3/NOS1^{-/-} myocytes, suggesting that SR calcium reuptake function (mediated by SERCA2) is not responsible for the observed abnormalities in Ca²⁺ handling in NOS^{-/-} (FIG. 1E). Additionally, Na/Ca exchanger (NCX) function was not different between WT and NOS1^{-/-} myocytes (FIG. 1F). These results support the hypothesis that diastolic Ca²⁺ leak from the sarcoplasmic reticulum (SR) may underlie the altered Ca²⁺ handling in NOS1 deficient mice.

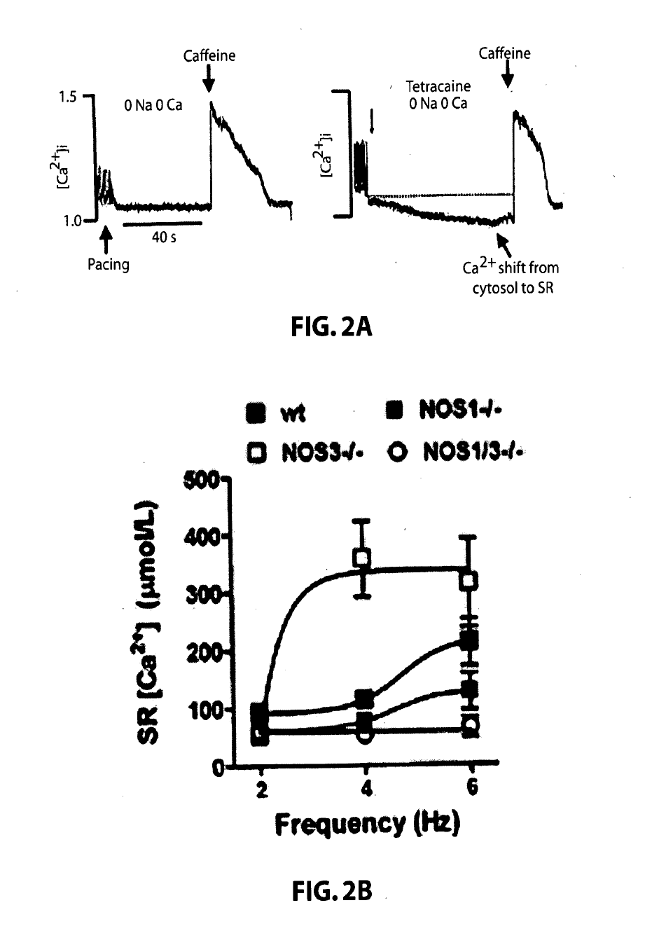

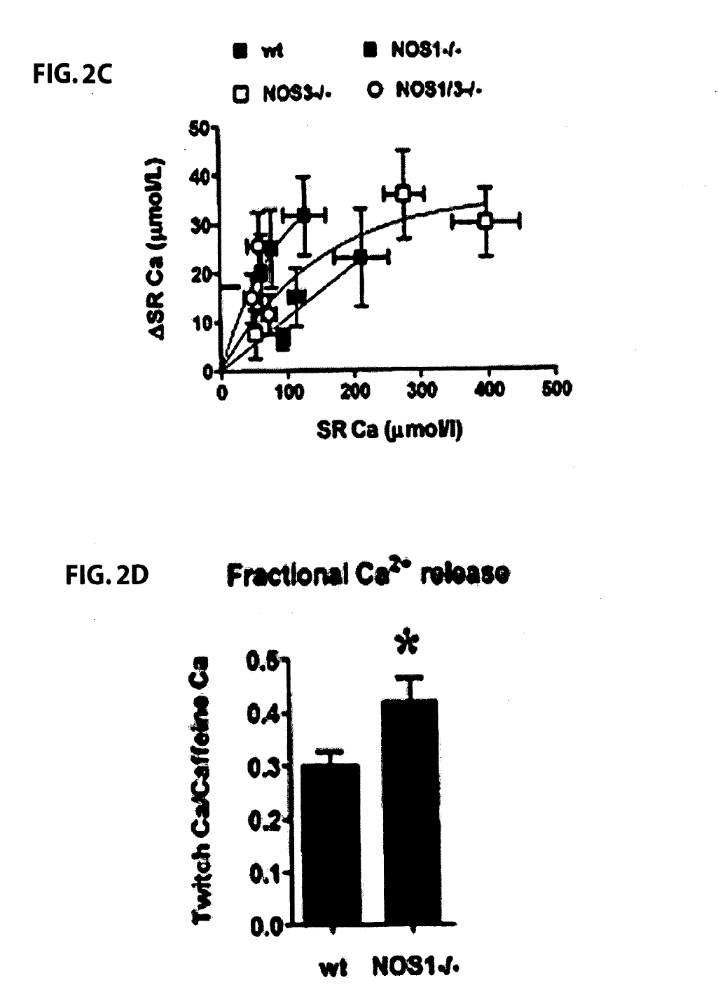

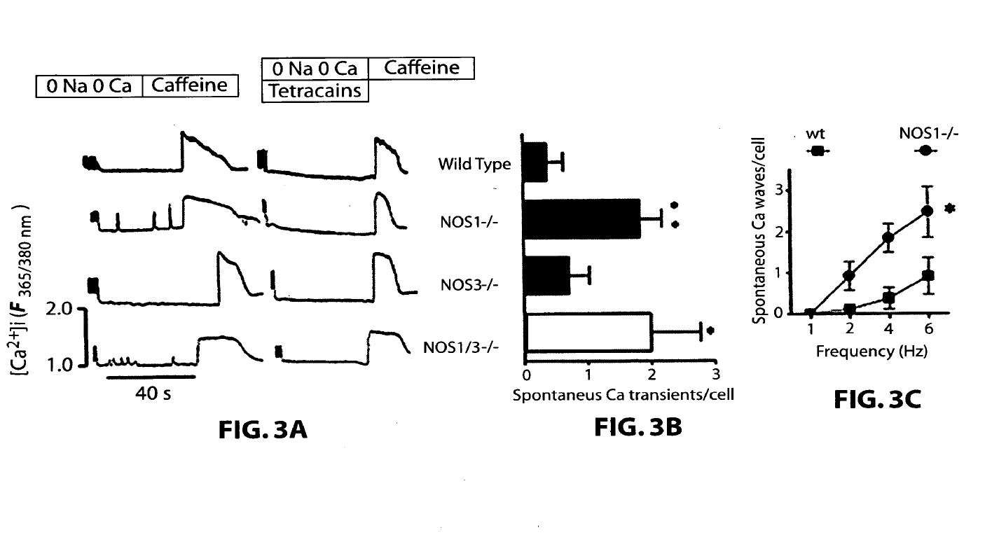

Assessment of SR Ca²⁺ content and diastolic Ca²⁺ leakage. To address this possibility, SR Ca²⁺ leak was directly measured using an established protocol (Shannon, T. R., et al. (2002). Circ. Res. 91:594-600) (FIG. 2A). This measurement indicates that deficiency of NOS1, whether in the NOS1^{-/-} or NOS3/NOS1^{-/-} myocytes exhibited substantially increased leak for a given Ca²⁺ load compared with WT. NOS1 deficiency is also associated with reduced SR Ca²⁺ content in the NOS1^{-/-} and NOS3/NOS1^{-/-} was decreased compared to wild type, while at 4 Hz it was increased in NOS3^{-/-} myocytes (FIG. 2A). Importantly, the fractional Ca²⁺ release was increased in NOS^{-/-} cardiomyocytes, denoting increased sensitivity of RyR to Ca²⁺ (FIG. 3D). This protocol revealed another important phenotype associated with myocyte electrical instability. During the period of 0Na⁺/0Ca²⁺ treatment, both NOS1^{-/-} and NOS3/NOS1^{-/-} myocytes displayed spontaneous Ca²⁺ transients (FIG. 3A). These transients were due to RyR2 opening as they were abolished by tetracaine treatment and occurred in the absence of extracellular Na⁺ or Ca²⁺, which impairs NCX activity.

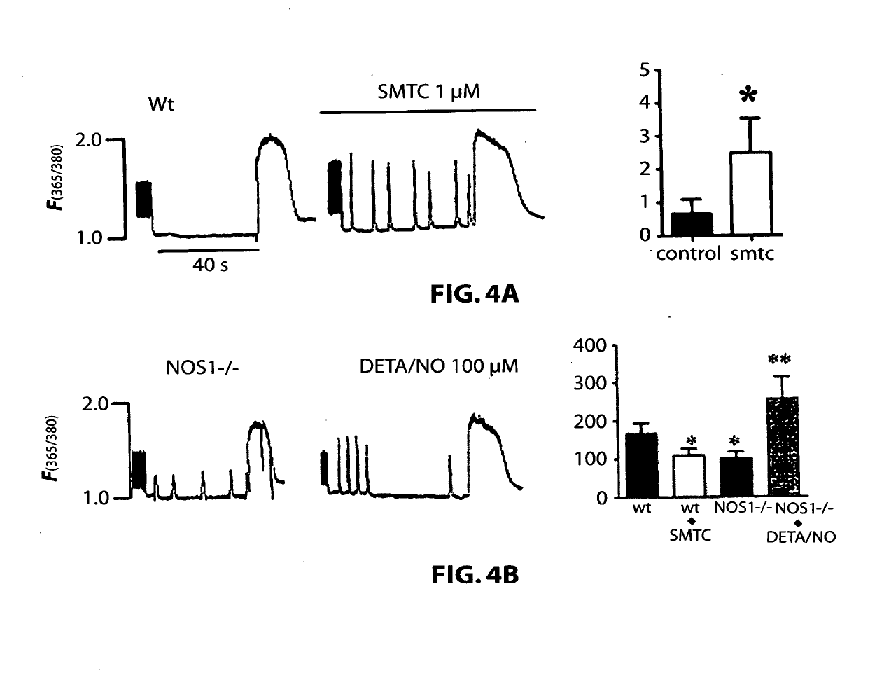

Next, whether the pharmacological inhibition of NOS1 with S-Methyl-thiocitrulline (SMTC; 1 μM), a specific NOS1 inhibitor (Babu, B. R., and Griffith, O. W. 1998. J. Biol. Chem. 273:8882-8889) mimicked the effects of the genetic deletion, was tested. This maneuver produced a decrease in the SR Ca²⁺ content (FIG. 4A) and induced the appearance of Ca²⁺ waves (FIG. 4B). In order to reverse this phenotype, NOS1^{-/-} myocytes were treated with a NO donor. The cells were incubated with DETA/NO (100 μM) for 5 mm and after this period, paced at 4 Hz for the leakage protocol. DETA/NO increased SR content but did not reduce the diastolic Ca²⁺ waves (FIG. 4B).

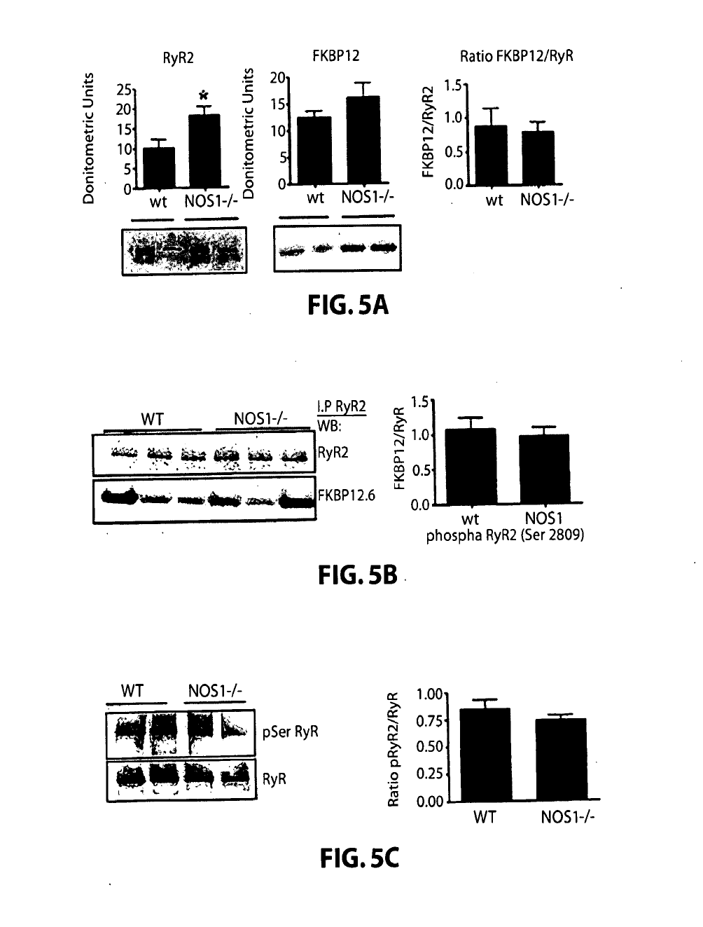

RyR2 phosphorylation and binding to FKBP12. Whether NOS1 disruption and the resulting Ca²⁺ leak were associated with alterations in the abundance of SR Ca²⁺ handling, was examined. Western blotting revealed that RyR2 expression was increased in the NOS1 hearts. FKBP12.6 was coordinately up-regulated, preserving a normal ratio between FKBP12.6 and RyR2 abundance in NOS1^{-/-} hearts (FIG. 5A). Also, coimmunoprecipitation experiments were performed to confirm that the stoichiometry of FKBP: RyR2 was not altered. There was no significant difference in the amount of FKBP bound to RyR2 in both strains (FIG. 5B).

Since RyR2 phosphorylation alters channel activity and binding to FKBP12.6, phosphorylation status in WT and NOS1^{-/-} animals was further investigated using an specific antibody against the phosphorylated Ser 2809 (FIG. 5C). The ratio of phospho RyR to total RyR was no different among both groups (n=4). No significant changes were found in calsequestrin, phospholamban (PLB), the L-type calcium channel, and SERCA2a. Only NCX was found to be significantly up-regulated.

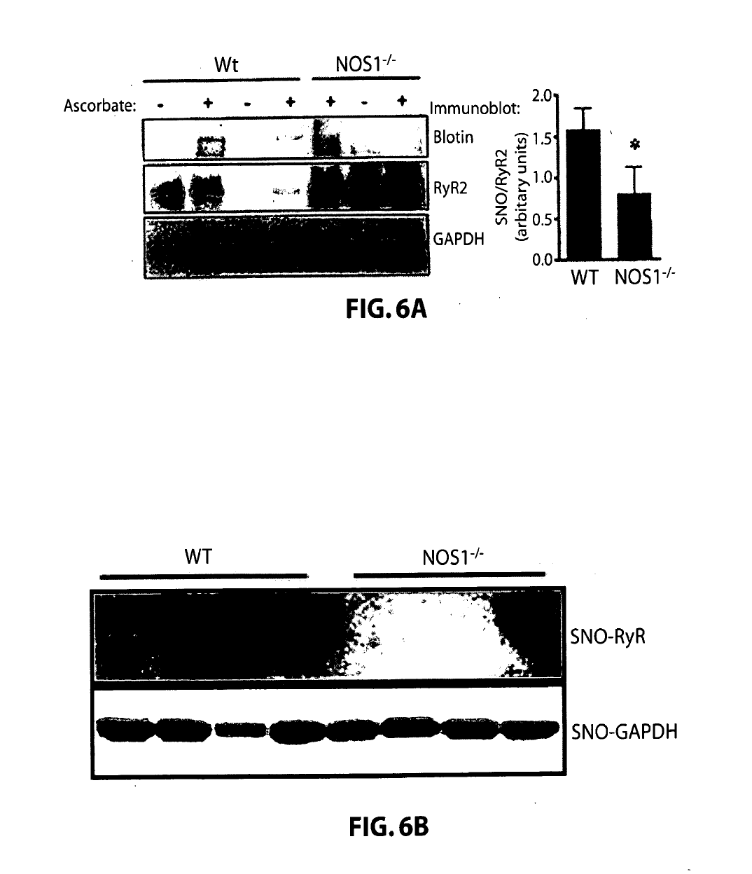

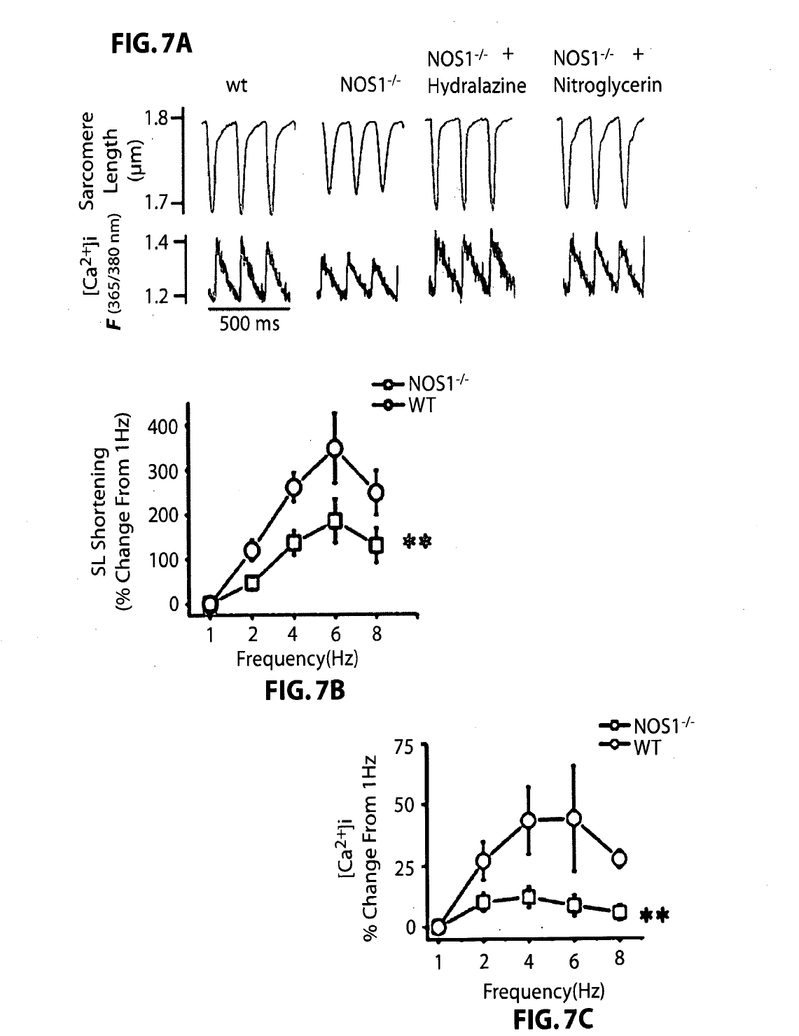

S-nitrosylation of RyR2. Finally, the degree of S-nitrosylation of RyR2 was evaluated, since this modification has been shown in electrophysiological experiments to alter the open probability of the channel. For this purpose, the biotin switch (FIG. 6A) was performed. With this assay, decreased degree of S-nitrosylation of RyR2 from the NOS1^{-/-} mice compared to Wt was found. This result was further confirmed by submitting a different set of hearts to the biotin switch except that this time, the biotinylated proteins were selectively pulled down with streptavidin-agarose (FIG. 7B). This methodology also showed markedly decreased S-nitrosylation of RyR2 from NOS1^{-/-} hearts. As a control, GAPDH, a well known S-nitrosylated protein, was analyzed. This was equally nitrosylated in both strains, highlighting the importance of the subcellular localization for the regulation of nitrosylation.

Discussion: The major new finding of the present study was that NOS1 was responsible for endogenous nitrosylation of the cardiac ryanodine receptor. RyR2 from NOS1 deficient mice was hyponitrosylated, and excitation-contraction coupling in these myocytes was marked by diastolic Ca²⁺ leak Importantly, this leakage causes not only depressed cardiac contractile reserve but also pro-arrhythmic spontaneous Ca²⁺ waves, which have been associated with sudden cardiac death. These findings demonstrated the endogenous regulation of the RyR2 by a specific NOS isoform and offer novel insights into pathophysiology of cardiac injury and arrhythmias.

In lipid bilayer preparations both RyR1 and RyR2 are nitrosylated at specific cysteine residues. The presence of NOS1 in the cardiac sarcoplasmic reticulum in proximity to RyR2 was demonstrated. Whether or not NOS1 is required for endogenous RyR2 nitrosylation and the linkage of this to cellular pathophysiology was heretofore unknown. The present results establish that indeed NOS1 is the NOS isoform required for RyR2 nitrosylation and NOS1 deficiency causes several key pathophysiologic features: diastolic Ca²⁺ leak and spontaneous diastolic Ca²⁺ waves. The appearance of these aberrant waves in diastole are the trigger for delayed after-depolarizations.

These characteristics have been observed, in certain mutations of RyR (Ter Keurs, H. E., and Boyden, P. A. 2007. Physiol Rev. 87:457-506) that include cysteine residues (Liu, N., 2006. Circ. Res. 99:292-298) and also in the phenotype of calsequestrin—(Knollmann, B. C., et al 2006. J. Clin. Invest 116:2510-2520), junctin (Yuan, Q., et al 2007. Circulation 115:300-309) and FKBP12.6 deficient mice (Wehrens, X. H., et al 2003. Cell 113:829-840). The cardiomyocytes of these animals display diastolic Ca²⁺ leak, spontaneous SR Ca²⁺ release triggered beats and increased mortality.

Several lines of evidence implicate a role for oxidative stress in the modulation of RyR2 activity. In NOS1-deficient mice, an increase in reactive oxygen species was shown (Khan, S. A., et al. 2004. Proc. Natl. Acad. Sci. U.S.A 101:15944-15948), and oxidative stress may oxidize reactive thiols on RyR2. In a canine model of heart failure, an increase in RyR oxidation and Ca²⁺ leak was observed, and corrected by antioxidant treatment (Yano, M., et al 2005. Circulation 112:3633-3643). In this sense, S-nitrosylation may prevent oxidation of reactive thiols of the channel, which is known to induce cross-linking between the subunits of RyR and increase the open probability of the channel and NO is able to prevent this. In the canine heart, RyR2 is endogenously S-nitrosylated and importantly, in a manner that is competitive with thiol oxidation. In this manner, NOS-1 derived nitrosylation may control the basal redox state of the channel. Furthermore, it may be that the tonic NO production in the SR may keep RyR in the closed state (Zahradnikova A., et al. 1997. Cell Calcium 22:447-454), what is also been observed with some NO donors (Hart J. D. and Dulhunty, A. F. 2000. J. Membr. Biol. 173:227-236; Suko, J., et al. 1999. Biochim. Biophys. Acta 1451:271-287).

Whereas in a physiological situation, like muscle activity, S-nitrosylation of the RyR may produce a reversible activation (required, for instance, during f3 adrenergic activation), in the pathological state, chronic RyR oxidation increases RyR gating in a more sustained, less reversible manner. In this case, leading to partial depletion of the SR and increasing the predisposition to ventricular arrhythmias.

Premature death in NOS1 and NOS3/NOS1 deficient mice (Barouch, L. A., et al. 2003. J. Mol. Cell Cardiol. 35:637-644) was discussed and that after myocardial infarction, the survival is dramatically decreased in NOS1 knock-out mice (Saraiva, R. M., et al. 2005. Circulation 112:3415-3422). It has been described that pharmacological blockade of NOS1 increases ventricular fibrillation in models of ischemia-reperfusion, effect that is reversed by NO donors.

On the other hand, NOS1 is up-regulated after myocardial infarction and translocates from the SR to the plasma lemma. This may inhibit the Ca²⁺ influx from the plasma membrane but at the same time disrupt the Ca²⁺ storage in the SR, as shown here. Furthermore, this disruption in Ca²⁺ homeostasis is closely linked to ventricular tachycardia, arrhythmias sudden cardiac death (Kannankeril, P. J., et al 2006. Proc. Natl. Acad. Sci. U.S.A 103:12179-12184; Paavola, J. et al. 2007. Heart J. 28: 1135-1142).

In summary, NOS1 deficiency causes diminished RyR2 S-nitrosylation, which in turn leads to increased diastolic Ca²⁺ and reduced intra SR Ca²⁺ content. This leakage leads to decreased contractility and increased electrical instability, key features of heart failure. Together these findings establish the importance of endogenous RyR2 S-nitrosylation mediated by NOS1, and provide mechanistic insights whereby NOS1 deficiency may lead to both depressed myocardial contractility as well as to sudden cardiac death.

### Example 2

### Hydralazine and Nitroglycerin Restore Impaired Excitation-Contraction Coupling Associated with Nitroso-Redox Disequilibrium

To test the hypothesis that organic nitrates and hydralazine improve NO bioavailability and reactivity, ameliorate NO/redox disequilibrium, and restore impaired E-C coupling. The effects of these drugs were examined, alone as well as in combination on ROS production by XOR and NADPH OXIDASE (NOX), E-C coupling, and Ca²⁺ cycling in vitro using a murine NOS1 knockout as a model of nitroso-redox disequilibrium.

### Materials and Methods:

Animal Model: 2-3 months old (n=25) C57BL/6 mice (WT, Jackson Laboratories, Bar Harbor, Me.) were studied and transgenic mice with homozygous deletions for NOS1 (2-3 months old, n=41) bred on a C57BL/6 background (originally bred in the laboratory of Dr. Mark Fishman, Massachusetts General Hospital). All protocols and experimental procedures were approved by the Institutional Animal Care and Use Committee of The Johns Hopkins University School of Medicine.

Isolated Myocytes Preparation: Cardiac myocytes were isolated and prepared from mouse hearts as described in detail by Khan et al. (Khan S. A. et al. Circ Res 2003;92:1322-1329). Sarcomere length (SL) and [Ca²⁺]ᵢ were measured simultaneously in myocytes stimulated at 1, 2, 4, 6 and 8 Hz. Experiments were then repeated after 5 minutes of incubation with hydralazine (0.01 mmol/L, Sigma, St. Louis, Mo.), nitroglycerin (0.01 mmol/L; American Regent Laboratories, Inc. Shirley, N.Y.), diethylenetriamine/nitric oxide adduct (DETA/NO, 0.1 mmol/L, Sigma, St. Louis, Mo.), allopurinol (0.1 mmol/L, Ben Venue Labs, Inc. Bedford, Ohio) hydralazine plus nitroglycerin and hydralazine plus DETA/NO. All experiments were conducted at 37° C. SL was recorded with an IonOptix iCCD camera. Change in average SL was determined by Fast Fourier Transform analysis of the Z-line density trace to the frequency domain, and SL Shortening was calculated as follows: (diastolic SL—systolic SL)/diastolic SL. Intracellular calcium, was measured using the Ca²⁺-sensitive dye Fura-2 and a dual-excitation spectrofluorometer (IonOptix), alternately excited with a xenon lamp at wavelengths of 365 and 380 nm The emission fluorescence was reflected through a barrier filter (510±15 nm) to a photomultiplier tube. The Fura-2 fluorescence ratio, i.e., the ratio of the photon live count detected by the excitation at 365 nm compared with 380 nm, represents [Ca²]ᵢ.

In order to assess calcium stores, caffeine (10 mmol/L) was rapidly infused after a 10 second pause following steady-state pacing at 1 and 4 Hz. In order to calculate the [Ca²⁺]ᵢ of caffeine pulse was normalized as follows:

([Ca²⁺]i at caffeine−[Ca²⁺]i at pacing)/[Ca²⁺]i at pacing×100.

Experiments were then repeated after incubating the cells with previously mentioned drugs.

Assessment of SR Ca²⁺ leakage: Calcium leakage was assessed as described by Shannon et al (Shannon T. R. et al. Circ Res 2002; 91:594-600). Ventricular myocytes were paced by field stimulation at the different frequencies in normal Tyrode until cellular Ca²⁺ transients reached a steady state. After the last pulse, the superfusing solution was rapidly switched to 0Na⁺-0Ca²⁺(Na⁺ replaced by Li⁺) Tyrode with or without 1 mmol/L tetracaine. In the control condition, [Ca²⁺]ᵢ was monitored while 0Na⁺-0Ca²⁺ Tyrode buffer was applied at least 40 seconds, rapid pulse of 10 mm/L caffeine was added to cause SR Ca²⁺ release. The difference between the basal and the peak total cytosolic [Ca²⁺]ᵢ in the presence of caffeine is, therefore, [Ca²⁺]τ. In the test condition, 0Na⁺0Ca²⁺ Tyrode was applied with tetracaine. In this condition, the leakage is blocked and the difference in [Ca²⁺]τ between tetracaine and control condition is diastolic leakage. To calculate [Ca²⁺]τ in the SR, the total SR volume was considered 3% of the cell volume and the cytosol volume, 65% of the cell volume.

NOX-dependent superoxide production: NADPH-dependent superoxide production was measured in heart homogenates using lucigenin (5 μmol/L)-enhanced chemiluminescence (β-NADPH 300 μmol/L; room temperature) on a microplate luminometer (Veritas, Turner Biosystems, Sunnyvale, Calif.). Briefly, proteins were diluted in modified Krebs-HEPES buffer. NADPH and lucigenin were added to wells just before reading. Chemiluminescence readings were expressed as integrated light units. Experiments were performed in the presence of hydralazine and nitroglycerin at different concentrations (0.01, 0.1 and 1 mmol/L). Experiments also were performed in the presence of the flavoprotein inhibitor diphenyleneiodonium (DPI, 0.01 mmol/L).

Xanthine Oxidoreductase activity: XOR activity was investigated by measuring uric acid and superoxide production. Heart homogenates were passed through a Sephadex G-25 column (GE Healthcare, Piscataway, N.J.) and XOR-dependent superoxide production was measured using Amplex red (Molecular Probes, Eugene, Oreg.) (0.05 mmol/L), horseradish peroxidase (HRP, 0.2 U/ml) and xanthine (0.1 mmol/L). In the assay, xanthine oxidase catalyzes the oxidation of xanthine to uric acid and superoxide. Superoxide spontaneously degrades to hydrogen peroxide (H₂O₂), and this, in the presence of HRP, reacts with Amplex Red to generate resurofin. Resurofin fluorescence was measured at 590 nm with excitation at 530-560 nm In parallel, the processed effluent was assessed spectrophotometrically at 295 nm for uric acid. The reaction mixture in 50 mM phosphate buffer, pH 7.4, contained 50 μl of the effluent, EDTA-Na₂ (1 mmol/L), xanthine (0.2 mmol/L). Both assay plates were incubated at 37° C. for 30 minutes in the presence or absence of allopurinol, hydralazine and nitroglycerin at increasing concentrations. The obtained values were plotted against a standard curve with known concentrations of XOR.

NADPH oxidase dependent and XOR dependent ONOO— production: NADPH oxidase and XOR dependent ONOO— production was measured in heart homogenates using p-aminophenyl fluorescein (APF) as indicator. Proteins were diluted in modified Krebs-HEPES buffer and incubated with NADPH or xanthine for 30 minutes at 37° C. APF was added just before the fluorescence readings, measuring at 515 nm with excitation at 490 nm. Experiments were performed in the presence of different concentrations of hydralazine and nitroglycerin.

Statistical Analysis: Data are reported as mean±SEM. Statistical significance was determined by one-way or two-way ANOVA and Student-Newman-Keuls post hoc test. The null hypothesis was rejected at P<0.05.

### Results

Cardiac Myocytes Force Frequency Response: SL and [Ca²⁺]ᵢ were measured in isolated cardiac myocytes from WT and NOS1^{-/-} mice during stimulation at 1, 2, 4, 6 and 8 Hz. Baseline SL shortening and [Ca²⁺]i were similar at 1 Hz (Table 1). NOS1^{-/-} myocytes (n=14 hearts; 1 to 3 cells per heart) exhibited depressed frequency-stimulated contractility and [Ca²⁺]ᵢ (FIG. 7B, 7C; P<0.01 versus WT, n=7; 1 to 3 cells per heart).

TABLE 1
Baseline Myocyte Characteristics at 1 Hz

| | WT | NOS1^{-/-} |
|---|---|---|
| Mice, n* | 7 | 14 |
| Diastolic SL, μm | 1.795 ± 0.015 | 1.820 ± 0.013 |
| SL Shortening, % | 1.74 ± 0.16 | 1.94 ± 0.23 |
| [Ca²⁺]ᵢ | 12.5 ± 2.13 | 14.9 ± 1.05 |

*Two to three cells were studied per heart.
SL Shortening indicates (diastolic SL − systolic SL)/diastolic SL;
[Ca²⁺]ᵢ, change in the ratio of the photon live count detected by excitation at 365 nm compared with 380 nm during contraction.

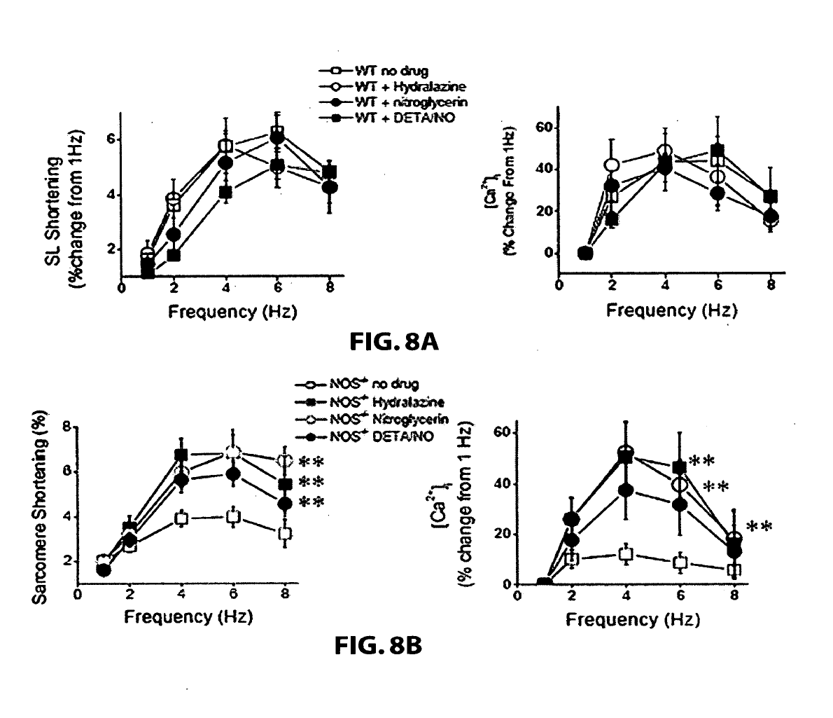

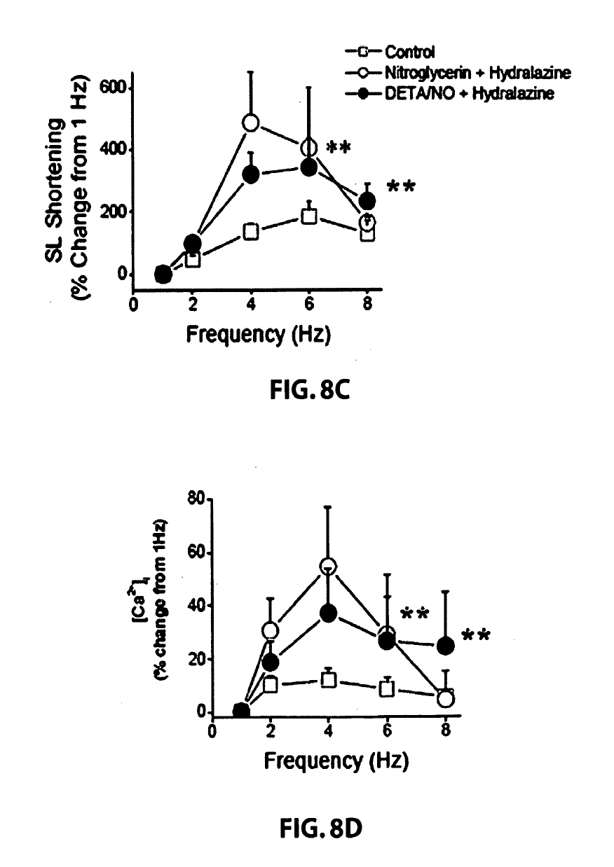

While hydralazine, nitroglycerin and DETA/NO had no effect in sarcomere shortening or calcium transients in wt myocytes (FIG. 8A), suppressed SL and [Ca²⁺]ᵢ responses in NOS1^{-/-} myocytes were restored towards levels similar to WT after incubation with hydralazine (n=8 hearts), nitroglycerin (n=6) and DETA/NO (n=10, 1-2 cells per heart with each drug; FIG. 8A, B; P<0.001, P<0.05, P<0.01 respectively). Next it was assessed whether drug combinations would have a synergistic effect. For this, NOS1^{-/-} myocytes were incubated with hydralazine plus nitroglycerin (n=5) and hydralazine plus DETA/NO (n=4). Interestingly, these drug combinations did not augment FFR to a greater extent than each drug alone (FIGS. 8C and 8D).

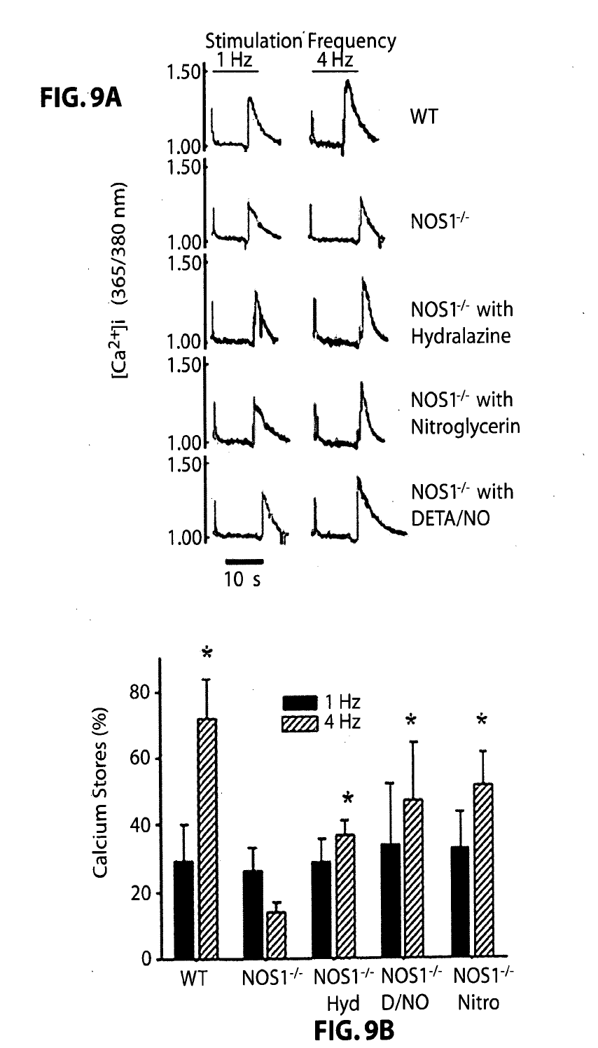

Sarcoplasmic Reticulum Ca²⁺: Since sarcoplasmic reticulum Ca²⁺ storage is a key determinant of the FFR, Ca²⁺ content was estimated by pacing the myocytes at 1 Hz and 4 Hz and after pause, rapidly infusing caffeine (FIG. 9). Ca²⁺ content at 1 Hz was similar in WT (% [Ca²+]i, 29.2±11.1, n=6) and NOS1^{-/-} mice (26.4±6.8, n=6). Increasing stimulation frequency to 4 Hz augmented SR Ca²⁺ content in WT cardiomyocytes (71.8±11.8%, n=6) but not in NOS1^{-/-} myocytes (13.7±3.1%, n=6, p<0.001 vs. WT). Sarcoplasmic reticulum Ca²⁺ content in NOS1^{-/-} cardiomyocytes was augmented by hydralazine (49.2±16.3%, n=6), nitroglycerin (42.8±6.5%, n=6) and DETA/NO (42.8±6.5% n=5, 1-2 cells per drug, FIG. 9B, P<0.05 for each drug, vs. control).

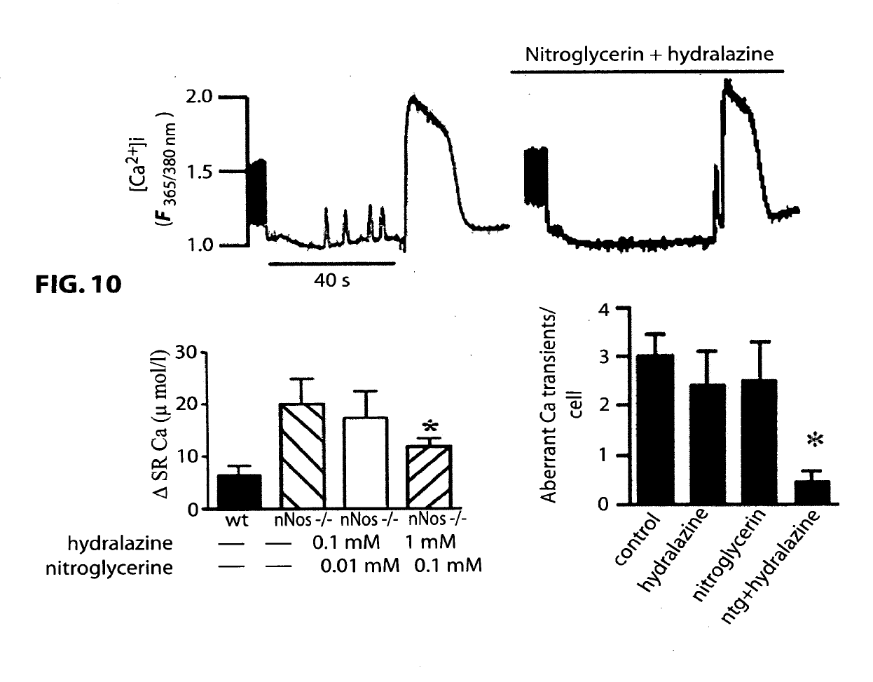

Assessment of SR Ca²⁺ leakage: A decrease in the Ca²⁺ content of SR stores may imply a decrease in Ca²⁺ influx, a decreased SERCA activity, and increased sodium/calcium exchanger activity or increased SR Ca²⁺ leak. It was investigated if the Ca²⁺ leak was responsible for the decreased Ca²⁺ storage. For this, an established protocol was applied using tetracaine, an RyR blocker (FIG. 10A). Diastolic Ca²⁺ leak was increased in NOS1^{-/-} myocytes compared to wt (FIG. 10B). This leakage was not abolished by either nitroglycerin (10 μmol/L) or hydralazine (100 μmol/L) alone. Conversely, a combination of both at a higher concentration (100 μmol/L nitroglycerin and 1 mmol/L hydralazine) was able to significantly reduce the Ca²⁺ leak (P<0.05, n=16 cells). Besides this effect, this combination was able to dramatically reduce spontaneous Ca²⁺ waves observed in NOS1^{-/-} myocytes (FIG. 10C). These waves are triggered by an increased RyR activity, since they are abolished by tetracaine.

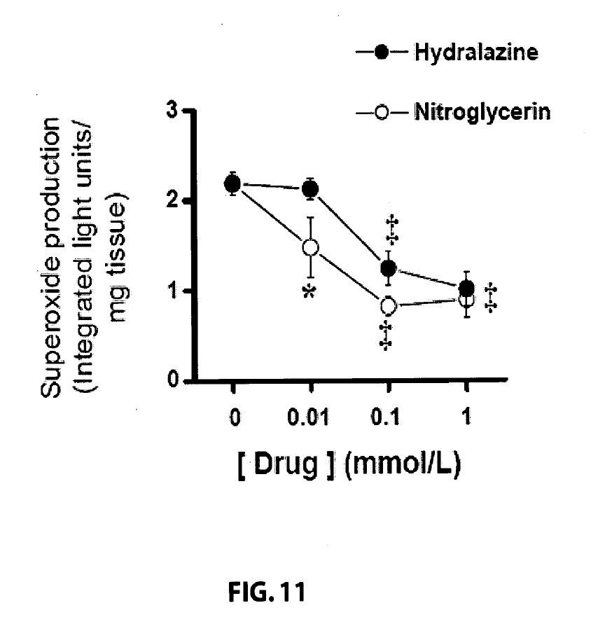

NOX-dependent superoxide production: In order to determine the molecular underpinnings for improved EC coupling in NOS1^{-/-} myocytes in response to the drugs used, XOR and NADPH oxidase activities were studied in the presence and absence of hydralazine and nitroglycerin in mice heart homogenates. NADPH oxidase activity of WT (n=3) and NOS1^{-/-} (n=4) hearts was similar (2.29±0.15 integrated light units/μg protein WT vs. 2.19±0.13 NOS1^{-/-}. Hydralazine (0.1-1 mmol/L, IC₅₀ 0.42±0.23 mmol/L) decreased NOX activity (FIG. 11, n=4, P<0.01 for both of concentrations vs. control) in NOS1−/− cardiac homogenates. Nitroglycerin (>0.01 mmol/L, IC₅₀ 0.013±0.06 mmol/L) also inhibited NOX dependent superoxide production (FIG. 11; n=4; P<0.05 for 0.01 mM, P<0.01 for 0.1 and 1 mmol/L vs. control).

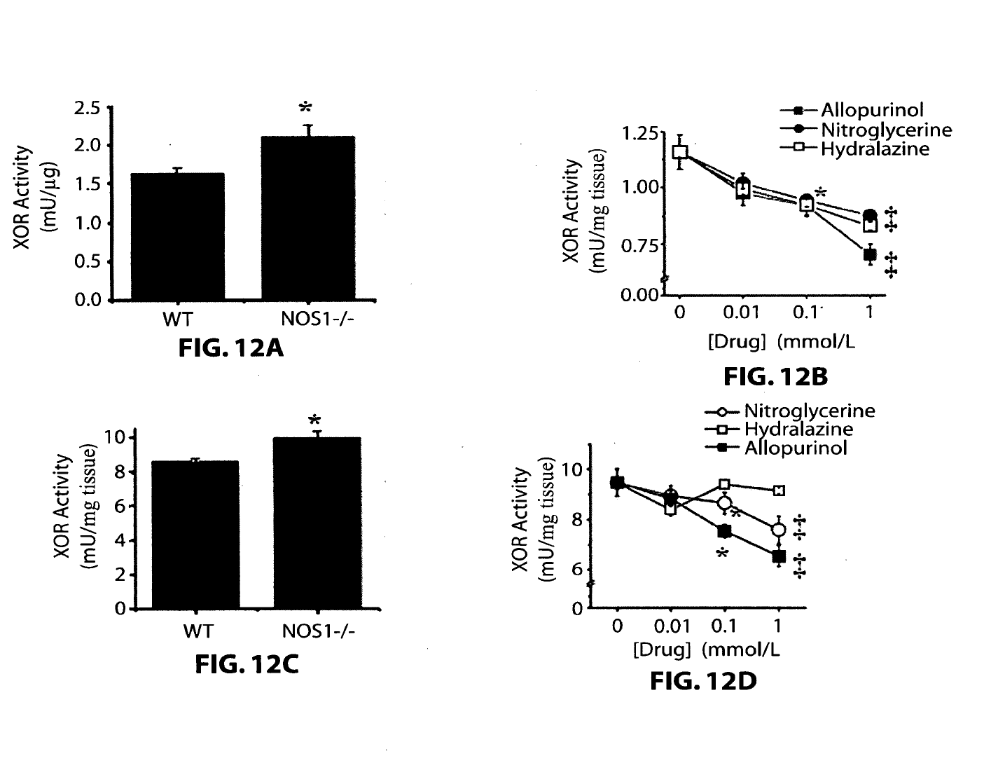

Xanthine Oxidoreducatse Activity: NOS1^{-/-} hearts (n=3) exhibited up-regulated XOR activity compared to WT (n=3) using the Amplex red detection of XOR-mediated ROS production assay (FIG. 12A; 2.1±0.18, 1.63±0.09 mU/μg protein, respectively; P<0.05), confirming previous findings in this mouse. Amplex red XOR activity was inhibited by allopurinol (>0.01 mmol/L, IC₅₀ 0.19±0.06 mmol/L, FIG. 11B; n=3) and hydralazine (IC₅₀ of 0.7±0.3 mmol/L, FIG. 12B; n=3) in NOS1^{-/-} cardiac homogenates. Interestingly, nitroglycerin (IC₅₀ 1.0±0.4 mmol/L, FIG. 12B, n=3) also inhibited XOR.

Since the Amplex Red assay measures superoxide levels, reduced activity could also reflect increased O₂⁻ scavenging. To address this, uric acid conversion was measured as a direct measurement of enzymatic activity. With this assay NOS1^{-/-} hearts (n=3) again exhibited increased XOR activity compared to WT (n=3) (FIG. 12C; 9.8±0.46 vs. 8.5±0.21 mU/mg tissue, respectively, P<0.05). Allopurinol (>0.1 mmol/L) inhibited XOR enzyme activity in NOS1−/− mice (FIG. 12D, n=3). Interestingly, hydralazine did not, but nitroglycerin did display XOR-inhibitory activity in NOS1−/− (FIG. 12D, n=3).

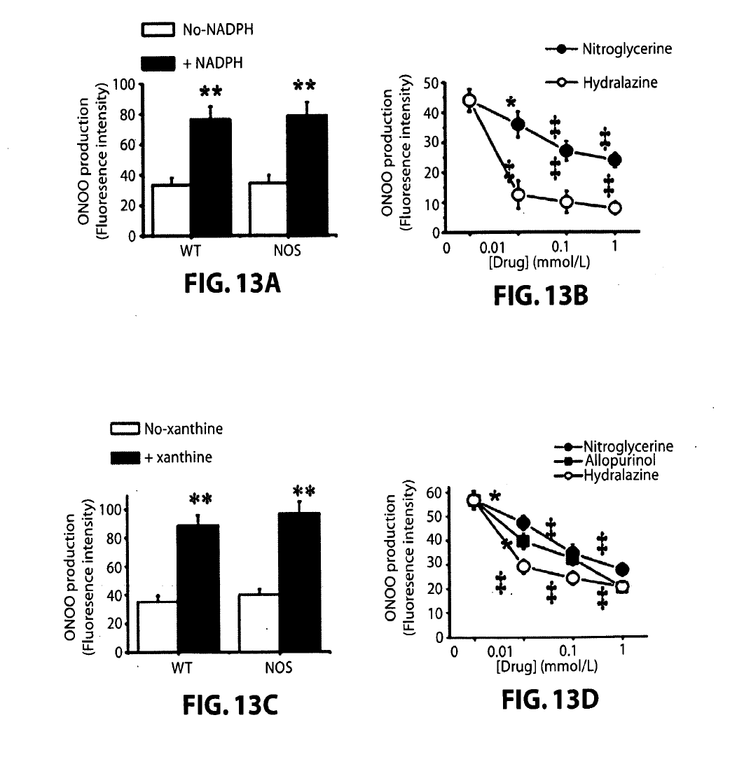

ONOO⁻ production: NOX-dependent peroxynitrite production was not different between WT (n=5) and NOS1^{-/-} mice (n=4) at baseline (32.7±3.8 vs. 34.1±3.7 fluorescence intensity/mg protein, respectively, FIG. 13A). Nitroglycerin decreased peroxynitrite production in NOS1^{-/-} heart extracts at concentrations above 0.01 mmol/L (IC₅₀ 1.3±0.4 mmol/L; FIG. 13B). Hydralazine inhibited more potently NOX-dependent peroxynitrite production (>0.01 mmol/L; IC₅₀ 0.019±0.03 mmol/L; FIG. 13D). Similar results were obtained using xanthine to stimulate XOR-dependent peroxynitrite production.

Discussion: The major new finding of this study is that hydralazine and nitroglycerin, alone and in combination, normalize the impaired force-frequency response and calcium cycling in NOS1^{-/-} cardiac myocyte. Hydralazine and nitroglycerin have important activity with regard to two major sources of cellular ROS production: NADPH oxidase (NOX) and XOR. Here it was also demonstrated that hydralazine inhibits NOX but not XOR, and scavenges ROS including ONOO⁻. Importantly, nitroglycerin inhibits both NOX and XOR, and also exhibits ROS scavenging capacity. Together, this regimen has the capacity to restore the disturbed NO— redox disequilibrium, which is a likely mechanism of restoration of E-C coupling.

Hydralazine is a peripheral vasodilator which has been used for many years in the treatment of essential hypertension and recently in heart failure. The basic mechanism of its effect on vasculature has been studied y but remains unknown. Here the effects of hydralazine on cardiomyocyte excitation-contraction coupling and oxidative stress were investigated. Hydralazine enhanced depressed NOS1−/− cardiomyocyte contractility and [Ca²⁺]ᵢ responses resulting from NO— redox imbalance. At a biochemical level, hydralazine exhibited O₂⁻ and ONOO⁻ scavenging capacity. While it is not clear whether O₂.⁻ scavenging is operative in vivo, given that the IC₅₀ of hydralazine (0.42±0.23 mmol/L) observed here is ˜50-fold higher than blood concentration of this drug (1.6 mg/L=0.008 mmol/L), it is possible that scavenging of ONOO⁻ produced by either NOX or XOR may occur, as the IC₅₀ for this activity is very close to the plasmatic levels of the drug. Actions of hydralazine to directly inhibit NOX and XOR cannot be excluded as hydralazine binds to vascular cells, cardiomyocytes and other cell types allowing it to reach relative high and stable local concentrations. In combination with nitroglycerin, hydralazine was able to reduce the diastolic Ca²⁺ leak, an effect caused by increased open probability of RyR. Calcium leakage is a salient feature of heart failure and has important consequences: depletes the SR of Ca²⁺, in a way that the contractile reserve is diminished and also has the potential trigger delayed after depolarizations (DAD). OS produces oxidation of RyR, increasing its diastolic leakage. In this sense, it is possible that nitroglycerin and hydralzine restore the redox state of the channel.

NO plays an important role in cardiac Ca²⁺ cycling and E-C coupling, influencing ion channels via S-nitrosylation. Importantly, deficiency of NOS1 from the cardiac SR contributes to depressed contractile reserve. In this study it was shown that nitroglycerin and NO donors augment cardiac contractility, an effect that could result from restored nitrosylation of ion channels like the ryanodine receptor. Indeed, it was shown that the combination of nitroglycerin and hydralazine was able to restore the normal activity of RyR.

The current results indicate that hydralazine, nitroglycerin, and DETA/NO affect contractility and Ca²⁺ cycling, including an increase of SR calcium stores, an affect attributable in all likelihood to the ROS scavenging capacity of these compounds.

In summary, the present data show novel mechanistic effects of hydralazine and nitroglycerin. The combination of these agents comprehensively inhibits two major ROS generating enzymatic systems, NOX and XOR, and also quenches O₂⁻ and ONOO⁻. The net cellular physiologic effect is a restoration of myocardial contractility and Ca²⁺ cycling towards normality, in part, by a normalization of the RyR activity. Thus, these drugs alone, and in combination, exert direct myocardial effects that are likely to lead to favorable functional and structural responses in the treatment of congestive heart failure.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the will allow the reader to quickly ascertain the nature of the technical disclosure. It is submitted with the understanding that it will not be used to interpret or limit the scope or meaning of the following claims.

What is claimed is:

1. A method of treating heart disease and heart disorders comprising: administering to a patient an agent comprising a nitric oxide (NO) donor or functional nitric oxide synthase molecule in a pharmaceutical composition, wherein the agent modulates ryanodine receptor (RyR) S-nitrosylation to normal levels as compared to a normal subject; and,
treating heart disease and heart disorders.

2. The method of claim 1, wherein the administration of the NO donor comprising agent stabilizes the diastolic calcium (Ca²⁺) levels to normal levels as compared to a normal subject.

3. The method of claim 1, wherein the agent is a nucleic acid molecule expressing a functional nitric oxide synthase in a patient or cardiac cells thereof.

4. The method of claim 1, wherein the agent comprises at least one of : organic compounds, inorganic compounds, polynucleotides, oligonucleotides, proteins, antisense molecules, siRNA, small molecules or combinations thereof.

5. A method of diagnosing a subject for nitric oxide synthase (NOS) deficiency comprising:
obtaining a sample from a patient;
screening the patient sample for nitric oxide synthase deficiency; and,
diagnosing a subject for nitric oxide synthase (NOS) deficiency.

6. The method of claim 5, wherein the patient sample is screened for levels of nitric oxide synthase, genetic differences in nitric oxide synthase nucleic acids, expression of nitric oxide synthase proteins and peptides and differences in peptide sequences as compared to a normal patient.

7. The method of claim 5, wherein the nitric oxide deficiency screening further comprises measuring ryanodine receptor (RyR) S-nitrosylation levels in a patient as compared to a normal subject.

8. The method of claim 7, wherein ryanodine receptor nitrosylation is measured by a biotin switch assay.

9. A method of screening and identifying agents which modulate nitric oxide in a cell or a sample from a patient comprising:
culturing a nitric oxide synthase deficient cell with a candidate agent; and,
measuring levels of nitric oxide and/or S-nitrosylation of ryanodine receptor; and,
comparing the levels of nitric oxide and/or S-nitrosylation of the ryanodine receptor to a normal cell; and,
screening and identifying agents which modulate nitric oxide in a cell or a sample from a patient.

10. The method of claim 9, wherein the nitric oxide synthase is neuronal nitric oxide synthase (NOS1).

11. The method of claim 9, wherein the agents modulate NADPH oxidase (NOX) activity and xanthine oxidoreductase (XOR) activity as compared to a normal control.

12. A method of screening and identifying agents which modulate nitric oxide in an animal comprising:
administering to the animal a candidate agent; and,
measuring levels of nitric oxide and/or S-nitrosylation of ryanodine receptor in a cell or sample from an animal; and,
comparing the levels of nitric oxide and/or S-nitrosylation of the ryanodine receptor to a normal cell or sample; and,
screening and identifying agents which modulate nitric oxide in an animal.

13. The method of claim 12, wherein the animal is a neuronal nitric oxide synthase (NOS1) deficient animal.

14. The method of claim 12, wherein the candidate agent stabilizes the diastolic to calcium (Ca²⁺) levels to normal levels as compared to a normal subject or animal.

15. The method of claim 12, wherein the candidate agents inhibit NADPH oxidase (NOX) activity and xanthine oxidoreductase (XOR) activity.

16. The method of claim 12, wherein the candidate agents equilibrate the nitroso-redox levels in a cell or patient to levels compared to a normal cell.

17. An isolated cell expressing a defective nitric oxidase synthase gene product or is deficient in nitric oxide synthase activity.

18. The isolated cell of claim 17, wherein the S-nitrosylation levels of the ryanodine receptor is hyper S-nitrosylated or hypo S-nitrosylated as compared to a normal cell.

19. The isolated cell of claim 17, wherein the cell is a mammalian cell comprising a vector expressing a defective nitric oxide synthase gene product wherein nitric oxide synthase activity is decreased as compared to a normal cell.

20. An isolated cell lacking a nitric oxide synthase gene product.

21. A cell comprising an expression vector having an inducible promoter operably linked to a polynucleotide comprising at least one of nitric oxide synthase polynucleotide, ryanodine receptor, NADPH oxidase, xanthine oxidoreductase, variants, mutants and fragments thereof.

22. A vector comprising an inducible promoter operably linked to a polynucleotide comprising at least one of nitric oxide synthase polynucleotide, ryanodine receptor, NADPH oxidase, xanthine oxidoreductase, variants, mutants and fragments thereof.

23. A method of treating cardiac arrhythmia or sudden cardiac death comprising: administering to a patient an agent comprising an NO donor in a pharmaceutical composition, wherein the agent modulates ryanodine receptor (RyR) S-nitrosylation to normal levels as compared to a normal subject; and,
treating cardiac arrhythmia

24. The method of claim 23, wherein the administration of the NO donor comprising agent stabilizes the diastolic to calcium (Ca²⁺) levels to normal levels as compared to a normal subject and prevents the leakage of calcium.

25. A stem cell comprising a nucleic acid molecule expressing a functional nitric oxide synthase gene product.

26. The stem cell of claim 25, wherein the nitric oxide synthase gene product modulates ryanodine receptor (RyR) S-nitrosylation to normal levels as compared to a normal heart cell.

27. The stem cell of claim 25, wherein the nitric oxide synthase gene product stabilizes the diastolic to calcium (Ca²⁺) levels to normal levels as compared to a normal heart cell and prevents the leakage of calcium.

28. The stem cell of claim 25, wherein the nitric oxide synthase gene product inhibits NADPH oxidase (NOX) activity and/or xanthine oxidoreductase (XOR) activity.

29. A method of preventing or treating a cardiac disease or disorder in a patient comprising:
administering to a patient a composition comprising at least one of a modulator of NADPH oxidase activity, xanthine oxidoreductase activity or nitric oxide donor in a therapeutically effective concentration; and,
preventing or treating a cardiac disease or disorder in a patient.

30. The method of claim 29, wherein the nitric oxide donor comprises nitric oxide enhancing agents, cyclic nitric oxide donors, heterocyclic nitric oxide donors, homocyclic nitric oxide donors, nitric oxide adducts, or substitutes, derivatives and variants thereof.

31. The method of claim 29, wherein the nitric oxide donor is diethylenetriamine/nitric oxide (DETA/NO).

32. The method of claim 29, wherein the NADPH oxidase activity modulator comprises hydralazine, nitroglycerin peptides, organic compounds, inorganic compounds, polynucleotides, oligonucleotides, proteins, antisense molecules, siRNA, or small molecules.

33. The method of claim 29, wherein the xanthine oxidoreductase activity modulator comprises nitroglycerin, allopurinol, peptides, organic compounds, inorganic compounds, polynucleotides, oligonucleotides, proteins, antisense molecules, siRNA, or small molecules.

34. The method of claim 29, wherein the composition comprises hydralazine, nitroglycerin and diethylenetriamine/nitric oxide (DETA/NO) therapeutically effective concentration.

35. The method of claim 29, wherein the ryanodine receptor is endogenously nitrosylated.

36. The method of claim 29, wherein the composition decreases reactive oxygen intermediates (ROS) and peroxynitrite (ONOO⁻) molecules as compared to a normal control.

37. A composition for treating cardiac disorders comprising hydralazine, nitroglycerin and diethylenetriamine/nitric oxide (DETA/NO) in a therapeutically effective concentration.

38. A method of diagnosing a patient having nitric oxide deficiency comprising:
obtaining a sample from a patient;
screening the sample for detection of nitric oxide or nitric oxide synthase;
determining the levels of nitric oxide in the sample as compared to a normal control; and,
diagnosing a patient having nitric oxide deficiency.

39. The method of claim 38, wherein the levels of nitric oxide and nitric oxide synthase are determined by methods comprising one or more of: immunoassays, Western blotting, PCR, hybridization assays, nitric oxide detection assays, electrochemical assays, fluorometric and colorimetric assays, or electroluminescent assays.

40. The method of claim 38, wherein the step of detecting and determining levels of nitric oxide or nitric oxide synthase comprises measuring S-nitrosylation of a ryanodine receptor as compared to a control.

41. The method of claim 40, wherein the ryanodine receptor is ryanodine receptor 2.

42. A method of diagnosing a patient suffering from a disorder associated with nitric oxide deficiency comprising
obtaining a sample from a patient;
assessing levels of nitrosylation of a ryanodine receptor as compared to a normal control; and,
diagnosing a patient suffering from a disorder associated with nitric oxide deficiency.

43. The method of claim 42, wherein nitrosylation of the ryanodine receptor is decreased as compared to a normal control.

44. The method of claim 43, wherein a decrease in S-nitrosylation of a ryanodine receptor 2 is diagnostic of a patient suffering from a disorder associated with nitric oxide deficiency.

45. The method of claim 42, wherein a disorder associated with nitric oxide deficiency comprises cardiac diseases, cardiac disorders or cardiac arrest.
